(19) United States
(12) Patent Application Publication
(10) Pub. No.: US 2009/0197922 A1
(43) Pub. Date: Aug. 6, 2009

(54) COMPOSITIONS AND METHODS FOR TREATING PULMONARY HYPERTENSION

(75) Inventors: Mardi Gomberg Maitland, Chicago, IL (US); Mark J. Ratain, Chicago, IL (US); Joe G.N. Garcia, Chicago, IL (US); Michael Maitland, Chicago, IL (US); Liliana Moreno-Vinasco, Chicago, IL (US)

Correspondence Address: FULBRIGHT & JAWORSKI L.L.P., 600 CONGRESS AVE., SUITE 2400, AUSTIN, TX, 78701, US

(73) Assignee: The University of Chicago, IChicago, IL (US)

(21) Appl. No.: 12/161,400

(22) PCT Filed: Jan. 24, 2007
(86) PCT No.: PCT/US07/60995

§ 371 Date: Nov. 18, 2008

Related U.S. Application Data
(60) Provisional application No. 60/761,612, filed on Jan. 24, 2006.
(60) Provisional application No. 60/833,934, filed on Jul. 28, 2006.

(51) Int. Cl. A61K 31/44

(52) U.S. Cl. 514/350

(57) ABSTRACT

Compositions and methods of the invention are related to treating pulmonary hypertension using a Raf kinase inhibitor, such as sorafenib. IQ a particular aspect, pulmonary hypertension is pulmonary arterial hypertension.

This application claims priority to U.S. Provisional Patent application Ser. No. 60/761,612 filed Jan. 24, 2006 and Ser. No. 60/833,934 filed Jul. 28, 2006, each of which is incorporated herein by reference in its entirety.

### BACKGROUND OF THE INVENTION

I. Field of the Invention

The invention described herein is related generally to medicine and particularly cardiac and pulmonary medicine. The invention is further related to therapeutic and prophylactic treatment of pulmonary hypertension and particularly pulmonary arterial hypertension.

II. Background

Pulmonary hypertension (“PH”) refers to a disease characterized by sustained elevations of pulmonary artery pressure (Rubin, 1997). Generally, a patient having a mean pulmonary artery pressure equal to or greater than 25 mm Hg with a pulmonary capillary or left atrial pressure equal to or less than 15 mm Hg is characterized as having PH or as symptomatic of PH. These parameters may be measured in the subject at rest by right-heart catheterization. Pulmonary arterial hypertension (“PAH”) includes idiopathic pulmonary arterial hypertension; familial pulmonary arterial hypertension; pulmonary arterial hypertension in the setting of connective tissue diseases (e.g., localized cutaneous systemic sclerosis (CREST syndrome), diffuse scleroderma, systemic lupus erythematosus, mixed connective tissue disease, and other less common diseases), portal hypertension, congenital left-to-right intracardiac shunts, and infection with the human immunodeficiency virus); and persistent pulmonary hypertension of the newborn. For a review of the mechanisms of disease related to PAH see Farber and Loscalso (2004), which is incorporated herein by reference in its entirety.

The World Health Organization (WHO) has classified pulmonary hypertension into groups based on known causes. WHO group I includes patients with PAH including those patients with Idiopathic PAH; Familial PAH, and Associated PAH, which is related to certain conditions including connective tissue diseases, congenital systemic-to-pulmonary-shunts, portal hypertension, HIV infection, drugs and toxins, glycogen storage disease, Gaucher's disease, hereditary hemorrhagic telangiectasia, hemoglobinopathies, myeloproliferative disorders, splenectomy, and others; PAH associated with significant venous or capillary involvement; and persistent pulmonary hypertension of the newborn. WHO group II includes patients with pulmonary venous hypertension. WHO group III includes patients with pulmonary hypertension associated with hypoxemia. WHO group IV includes patients with pulmonary hypertension due to chronic thrombotic disease, embolic disease or both. Finally, WHO group V includes patients with pulmonary hypertension due a variety of miscellaneous conditions.

The WHO also classifies pulmonary hypertension into functional groups based on their exercise capacity and symptoms. WHO class I includes patients with PAH without limitations of physical activity. WHO class II includes patients with PAH resulting in slight limitation of physical activity. WHO class III includes patients with PAH resulting in marked limitation in physical activity. WHO class IV includes patients with PAH that are unable engage in physical activity without manifesting symptoms.

As stated above, PH is also associated with disorders of the respiratory system and/or hypoxemia, including chronic obstructive pulmonary disease, interstitial lung disease, sleep-disordered breathing, alveolar hypoventilation disorders, chronic exposure to high altitude, neonatal lung disease and alveolar-capillary dysplasia (Humbert, 2004). PH is associated with chronic thrombotic disease, embolic disease or both, and with a variety of miscellaneous conditions.

Based on the inadequate therapies available, a need remains for additional safe and effective methods of treating and managing PH.

### SUMMARY OF THE INVENTION

The present invention includes the use of sorafenib, an inhibitor of multiple kinases important to angiogenesis (Raf-1, VEGFR-2, VEGFR-3, PDGFR-β) shown to have anticancer properties, and sorafenib like compounds as an effective agent or therapy for reducing pulmonary arterial pressure or a symptom of pulmonary arterial pressure. The inventors contemplate using other inhibitors of angiogenesis or of VEGFR for reducing pulmonary arterial pressure or a symptom of pulmonary arterial pressure. The invention concerns therapeutic and preventative compositions and methods related to the various WHO groups of pulmonary arterial hypertension (PAH) (e.g. group I that includes idiopathic pulmonary arterial hypertension (IPAH) and familial pulmonary arterial hypertension (FPAH)), and Associated PAH, with the others in WHO group I. In certain embodiments, PAH may be limited to a specific type of PAH. In certain embodiments the methods can include providing a VEGFR2 inhibitor (i.e., sorafenib like compounds) to a subject with pulmonary arterial hypertension (PAH), with symptoms of PAH, or at risk for PAH. The VEGFR2 inhibitor can include abt-869, arng706, AZD2171, bay57-9352, sorafenib, XL647, XL999, GW786034, bevacizumab, PKC412, AEE788, PTK787 (vatalanib), OSI-930, OSI-817, SU11248, AG-013736, ZK3-4709, quinazoline ZD6474, pyrrolocarbazole CEP-7055, or CP-547632.

The present invention, in some embodiments, concerns methods that include providing a kinase inhibitor, such as sorafenib, to a subject with pulmonary arterial hypertension (PAH), with symptoms of PAH, or at risk for PAH. The term “provide,” and other related forms of the term, is used according to its ordinary meaning of to supply or to furnish, which may be accomplished directly or indirectly. It is contemplated that the patient may be provided sorafenib or other kinase inhibitor directly, such as by administering or prescribing, for example, sorafenib or a pharmaceutically acceptable salt thereof, or indirectly, such as by administering or prescribing a sorafenib prodrug or a pharmaceutically acceptable salt thereof, such that the subject is effectively provided with sorafenib.

In some embodiments, the patient has a mean pulmonary artery pressure equal to or greater than 25 mm Hg with a pulmonary capillary or left atrial pressure equal to or less than 15 mm Hg, and/or a pulmonary vascular resistance greater than or equal to 2 Wood Units. Pulmonary Vascular Resistance (PVR) is the general pressure load against which the right ventricle must pump to push blood through the lungs. PVR is typically expressed in Wood units which is defined by the formula: PVR=(MPAP minus PCWP) divided by cardiac output (CO). Less than 2 Wood Units equals the PVR of an average healthy person, i.e., MPAP is about 20 mmHg; LAP is about less than or equal to 15 mmHg; CO is about 5 liters per minute. MPAP is the Mean Average Pulmonary Artery Pressure which is the average pressure in the pulmonary artery. MPAP can be calculated using the formula: [(2 times diastolic pulmonary artery pressure (DPAP) plus systolic pulmonary artery pressure (SPAP)] divided by 3, with the normal MPAP range typically being between 10 to 20 mmHg. PCWP is the Pulmonary Capillary Wedge Pressure which is an indirect measurement of pressure in the heart's left atrium which can be directly monitored during pulmonary catheterization. The normal PCWP range is typically between 5 to 15 mmHg. CO is the volume of blood ejected by the heart per minute and can be calculated by the formula: CO (in liters/minute) equals heart rate (in beats/minute) times stroke volume (in liters/beat). The normal CO range is between 4 to 8 L/min. Normal PVR is typically considered between 0.7 to 2.0 Wood units. These parameters may be measured in the subject at rest by right-heart catheterization.

The term “administering” is used according to its ordinary meaning of to dispense, furnish, supply, or give. In many embodiments of the invention, the subject may administer sorafenib to themselves or a medical practitioner may provide the sorafenib to the subject. The term “prescribing” is used according to its ordinary meaning of to advise or order the use of, and it is generally contemplated that a medical practitioner would prescribe the sorafenib to the subject, which the subject would then administer to themselves. It is contemplated that any embodiment in which sorafenib is provided to a subject may be implemented in the context of administering or prescribing sorafenib, a sorafenib prodrug, or a pharmaceutically acceptable salt thereof.

In further embodiments, it is contemplated that the subject is provided an amount of sorafenib that is effective to treat or prevent PAH. It is contemplated that the term “treat” is used according to its ordinary meaning of to deal with a disease or affection, a part of the body, or a person in order to relieve or cure. Patients who have PAH or symptoms of PAH may be treated according to methods of the invention. It is further contemplated that treatment may include management of PAH or PAH symptoms, which means that treatment allows PAH or PAH symptoms to be controlled. The term “prevent” is used according to its ordinary meaning of to preclude, stop, or hinder. It is contemplated that an “effective amount” is an amount that achieves the stated goal, which may be treatment and/or prevention of PAH. It is contemplated that in the context of treatment an effective amount produces a therapeutic benefit, which includes, but may not be necessarily limited to the following characteristics with respect to pulmonary arterial hypertension: reducing mean pulmonary pressure, increasing cardiac output/cardiac index measured by either thermodilution or Fick, improving timed walk distance (e.g., six-minute walk), improving metabolic equivalents (MET) (e.g., exercise treadmill test), reducing anginal pain frequency, reducing dyspnea, synocope, presyncope, symptoms of right heart failure including edema and ascites, preventing need for lung or heart transplant, reducing length of stay in intensive care, reducing length of stay in hospital, or prolonging life.

Typically, a patient or subject is assessed by using the six-minute walk test. The test is administered by preparing an unobstructed path of a known distance, e.g., path of 50 feet (100 feet round-trip). A chair may be placed at each end of path. The patient is typically instructed to walk at his/her own pace and stop to rest if needed and to cover as much distance as possible. Typically, one would look for improvement between pretreatment assessment and post treatment assessments. The six minute walk distance may also be compared to distances walked by a comparable healthy population, typically by using equations from a published study of healthy people of the same age group. Improvement in a patient's or subject's condition may be indicated by an increased six minute walk distance.

Another method of assessing a patient or subject is the determination of tricuspid valve regurgitation velocity. Tricuspid valve regurgitation velocity is typically assessed by an echocardiogram, in particular Doppler echocardiograms. The trained sonographer possesses the requisite knowledge to carry out the echocardiograms in association with assessment by a qualified physician or technician for assessment. A typical echocardiographic imaging protocol includes a parasternal long-axis view; parasternal short-axis views at the aortic valve, mitral valve, and left ventricular levels; and apical 4-chamber, 2-chamber, and long-axis views. Mitral, aortic, pulmonic and tricuspid valves can be imaged by color Doppler in multiple views to determine the degree of regurgitation. Measurements of various cardiac dimensions are performed according to American Society of Echocardiography convention. Pulmonary artery systolic pressure can be calculated utilizing a modified Bernoulli equation, PAP=4v²+RAP, where v=peak systolic velocity or tricuspid regurgitation jet recorded by continuous wave Doppler and RAP (right atrial pressure) is assumed to be 10 mmHg. Visual estimates of left ventricular ejection fraction can be made by integrating information from all views and left ventricular ejection fraction is considered normal if above 0.50. Improvement in a patient's or subject's condition may be indicated by a decrease in tricuspid valve regurgitation, as well a upgrading in the WHO functional class designation.

In certain embodiments, the subject's mean pulmonary artery pressure is reduced to less than 25 mm Hg It is specifically contemplated that the subject may experience a reduction in his mean pulmonary artery pressure of about, at least about, or at most about 1, 2, 3, 4, 5, 10, 15, 20, 25, 30, 35, 40, 45, 50, 55, 60, 65, 70, 75 or greater percent, or any range derivable therein, relative the value prior to treatment with sorafenib, therein, relative the value prior to treatment with sorafenib. Such measurements may be taken at rest using a right heart catheterization, though other methods may be used.

Therefore, the present invention specifically includes methods for treating or preventing PAH in a patient comprising providing to the patient an effective amount of sorafenib or a pharmaceutically acceptable salt thereof. Subjects and patients specifically include humans, in addition to other mammals.

In some methods of the invention may further include identifying a subject with PAH or symptoms of PAH or a patient at risk for PAH. This may be achieved by a number of ways known to those of skill in the art, including, but not necessarily limited to, taking a patient history, inquiring about family members with PAH, obtaining the level of pressure in the subject's pulmonary artery, identifying in the subject risk factors for PAH, assessing an electrocardiogram of the subject, assessing an echocardiogram of the subject, assessing pulmonary function tests (PFTs) of the patient, assessing a perfusion lung scan, a high resolution CT scan of the chest and assessing a right-heart cardiac catheterization with vasodilatory testing.

In some embodiments of the invention, the subject has been diagnosed as having Class I, II, III, or IV PAH according to guidelines used for class diagnosis, such as by the New York Heart Association or as having Class I, II, III, or IV, such as by the World Health Organization. See Rubin, 2004, which is hereby incorporated by reference. The term “severe PAH” refers to Class 3 or 4 or Class III or IV, according to the relevant guidelines.

In further embodiments of the invention, after the subject has taken or been given sorafenib or a sorafenib prodrug, he/she experiences a reduction in pulmonary pressure. In some methods of the invention the reduction is about, about at least or about at most a reduction of 5, 10, 15, 20, 25, 30, 35, 40, 45, 50, 55, 60, 65, 70, 75, 80, 85, 90 percent, or any range derivable therein, relative the pressure prior to sorafenib intake. In other embodiments, there is a fall of mean pulmonary artery pressure of at least about 10 mm Hg after treatment with sorafenib. It is contemplated that the fall may be about, at least about, or at most about, 10, 15, 20, 25, 30, 35, 40, 45, 50 mm Hg, or any range derivable therein, after or during sorafenib treatment.

In further embodiments, methods involve evaluating PAH in the subject before and/or after the subject has taken or been given sorafenib. This can be achieved by a number of ways that include, but are not necessarily limited to, having an electrocardiogram, an echocardiogram, pulmonary function tests (PFTs), a perfusion lung scan, other vasodilator testing, and/or a right-heart cardiac catheterization.

The present invention also relates to methods in which a subject is administered or prescribed multiple doses of sorafenib. The subject may take or be given 2, 3, 4, 5, 6, 7, 8, 9, 10, 11, 12, 13, 14, 15, 16, 17, 18, 19, 20, 25, 30, 35, 40, 45, 50, 100, 200, 300, 400, (twice daily—oncologic dose), 500 doses or more of sorafenib. It is also contemplated that a subject may be given or prescribed sorafenib indefinitely or for a set period of time, such as 1, 2, 3, 4, 5, 6, 7 days, 1, 2, 3, 4, 5, weeks, 1, 2, 3, 4, 5, 6, 7, 8, 9 10, 11, 12 months, and/or 1, 2, 3, 4, 5, 6, 7, 8, 9, 10 years or more, or any range derivable therein. Alternatively, it is contemplated that a patient continues to be prescribed or administered sorafenib until the subject can be classified as a Class 1 or 2 subject (or Class I or II), the six mile walk distance is increased, the symptoms of PAH are relieved or reduced (such as triscuspid valve regurgitation), the mean pulmonary artery pressure is reduced to less than about 25 mm Hg or there is an improvement with respect to PAH or a symptom of PAH, such as a relative decrease or increase in the value of a measurement of about, at least about, or at most about 2, 3, 4, 5, 6, 7, 8, 9, 10, 11, 12, 13, 14, 15, 16, 17, 18, 19, 20, 25, 30, 35, 40, 45, 50, 55, 60, 65, 70, 76, 80, 85, 90, 95, 100 or more percent or -fold, or any range derivable therein, as compared to a value of a measurement prior to or during sorafenib therapy.

In embodiments of the invention, a single dose of sorafenib is between about 50 mg and about 400 or 600 mg twice daily of sorafenib, whereas a dose refers to a single, uninterrupted administration of sorafenib. A dose may be about, at least about, or at most about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 mg or mg/kg, or any range derivable therein. It is contemplated that a dosage of mg/kg refers to the mg amount of sorafenib per kg of total body weight of the subject. It is contemplated that when multiple doses are given to a patient, the doses may vary in amount or they may be the same.

Sorafenib may be introduced into a subject by a number of ways that include, but are not necessarily limited to, orally, intravenously, intraarterially, or inhalation.

The present invention also covers methods that also include administering or prescribing at least a second PAH treatment (“secondary PAH treatment”). Other compounds or agents that have been used to treat PAH: include, but are not limited to the following: an anticoagulant (such as Coumadin or Warfarin), an calcium channel blocker (such as amlodipine, diltiazem, nifedipine, felodipine, isradipine, nicardipine, or verapamil), a prostacyclin (such as epoprostenol, treprostinil, iloprost), nitric oxide (only used in acute settings), a diuretic, a cardiac glycoside (digoxin), an endothelin antagonist (including non-selective inhibition with bosentan), a phosphodiesterase inhibitor (such as sildenafil), an endopeptidase inhibitor, a lipid lowering agent, a thromboxane inhibitor (such as terbogrel), or oxygen. It is contemplated that a combination of treatments that include sorafenib may be employed including investigational agents such as sitaxentan and ambrisentan (selective endothelin antagonist), and tadalafil (long acting phosphodiesterase inhibitor). A description of different therapies is provided in Badesch et al., 2004, which is hereby incorporated by reference.

In certain embodiments, the sorafenib or composition containing sorafenib, or a secondary PAH treatment may be administered or prescribed before, after, or during surgery. In some embodiments, the surgery is lung transplantation, in which case other treatment is not subsequently needed.

Other embodiments of the invention include a pharmaceutical composition comprising sorafenib or a pharmaceutically acceptable salt thereof and at least a second PAI treatment, such as those discussed above.

Any embodiment discussed with respect to one aspect of the invention applies to other aspects of the invention as well. It is further contemplated that embodiments discussed in the context of PAH may be applied in the context of PH, and vice versa.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a”, and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

So the above-recited features, advantages, and objects of the invention, as well as others, will become clear and can be understood in detail, more particular descriptions and certain embodiments of the invention briefly summarized above are illustrated in the appended drawings. These drawings form a part of the specification. It is to be noted, however, that the appended drawings illustrate or are associated with certain embodiments of the invention and therefore are not to be considered limiting in their scope.

FIGS. 1A-1B FIG. 1A Shows pulmonary arterial pressure (mm Hg) of Dahl Salt Sensitive (SS) rats under normoxic conditions, hypoxic conditions (10% FiO₂), hypoxic conditions and SU5416 administration, hypoxic conditions and sorafenib administration, and hypoxic conditions and SU5416 and sorafenib administration. FIG. 1B shows effects of Sorafenib on pulmonary artery pressures and remodeling in rodent PH. Sorafenib prevents hypoxia+Su5416 induced pulmonary HT and remodeling.

FIG. 2 Shows right ventricle systolic pressure (mm Hg) of Dahl SS rats under normoxic conditions, hypoxic conditions, hypoxic conditions and SU5416 administration, hypoxic conditions and sorafenib administration, and hypoxic conditions and SU5416 and sorafenib administration.

FIG. 3 Shows hematocrit % in Dahl SS rats under normoxic conditions, hypoxic conditions, hypoxic conditions and SU5416 administration, hypoxic conditions and sorafenib administration, and hypoxic conditions and both SU5416 and sorafenib administration.

FIGS. 4A-4B Show right ventricular/left ventricular+septum ratio values, in Dahl SS rats under normoxic conditions, hypoxic conditions, hypoxic conditions and SU5416 administration, hypoxic conditions and sorafenib administration, and hypoxic conditions and SU5416 and sorafenib administration. Sorafenib prevented hypoxia+SU5416 development of right heart hypertrophy (RV/LV+Septum) in Dahl SS rats.

FIG. 5 Shows mean blood pressure (mm Hg) of Dahl SS rats under normoxic conditions, hypoxic conditions, hypoxic conditions and SU5416 administration, hypoxic conditions and sorafenib administration, and hypoxic conditions and SU5416 and sorafenib administration.

FIG. 6 Shows echocardiographic data on the calculated left ventricle mass (grams) of Dahl SS rats under normoxic conditions, hypoxic conditions, hypoxic conditions and SU5416 administration, hypoxic conditions and sorafenib administration, and hypoxic conditions and SU5416 and sorafenib administration.

FIG. 7 Shows echocardiographic data on the calculated aortic cardiac output (1/min) of Dahl SS rats under normoxic conditions, hypoxic conditions, hypoxic conditions and SU5416 administration, hypoxic conditions and sorafenib administration, and hypoxic conditions and SU5416 and sorafenib administration.

FIG. 8 Shows echocardiographic data on the calculated pulmonary arterial pressure gradient (mm Hg) of Dahl SS rats under normoxic conditions, hypoxic conditions, hypoxic conditions and SU5416 administration, hypoxic conditions and sorafenib administration, and hypoxic conditions and SU5416 and sorafenib administration.

FIG. 9 Shows echocardiographic data on the calculated right ventricle free wall thickness of Dahl SS rats under normoxic conditions, hypoxic conditions, hypoxic conditions and SU5416 administration, hypoxic conditions and sorafenib administration, and hypoxic conditions and SU5416 and sorafenib administration.

FIG. 10 Shows echocardiographic data on the calculated pulmonary arterial pressure (mm Hg) of Dahl SS rats under normoxic conditions, hypoxic conditions, hypoxic conditions and SU5416 administration, hypoxic conditions and sorafenib administration, and hypoxic conditions and SU5416 and sorafenib administration.

FIG. 11 Shows echocardiographic data on the calculated right ventricle thickness (cm) of Dahl SS rats under normoxic conditions, hypoxic conditions, hypoxic conditions and SU5416 administration, hypoxic conditions and sorafenib administration, and hypoxic conditions and SU5416 and sorafenib administration.

FIGS. 12A-12B Show VEGFR-2 immunostaining of endothelial cells (arrows) occluding the lumen of a small pulmonary artery of SU5416 treated lung (rat) exposed to chronic hypoxia for 3 weeks (600×) (FIG. 12A). Smooth Muscle Cell alpha actin immunostaining of a patent intra-alveoloar pulmonary artery of SU5416 treated lung (rat) exposed to chronic hypoxia for 3 weeks. Note that the precapillary vessel has acquired a well-defined medial smooth muscle cell layer (600×) (FIG. 12B).

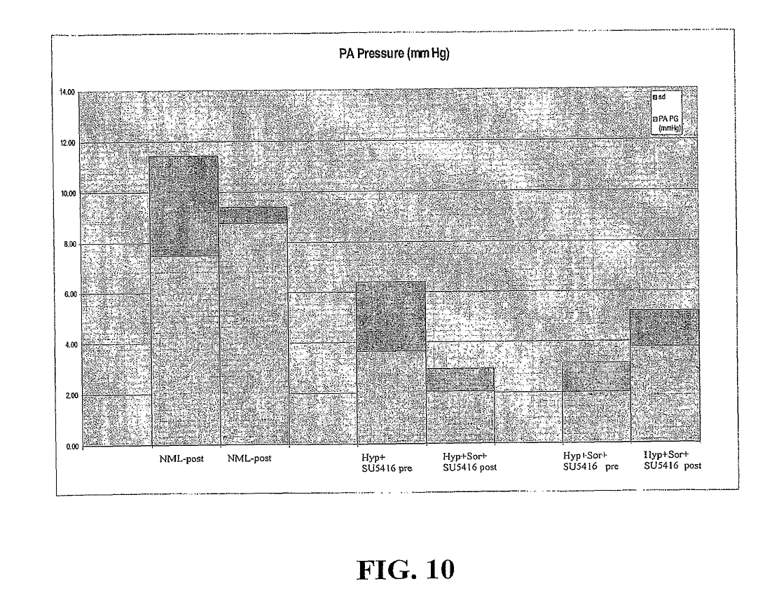

FIG. 13 Shows human pulmonary arteries demonstrating medial hypertrophy, intimal thickening, neomuscularization, and thrombin deposition (clot).

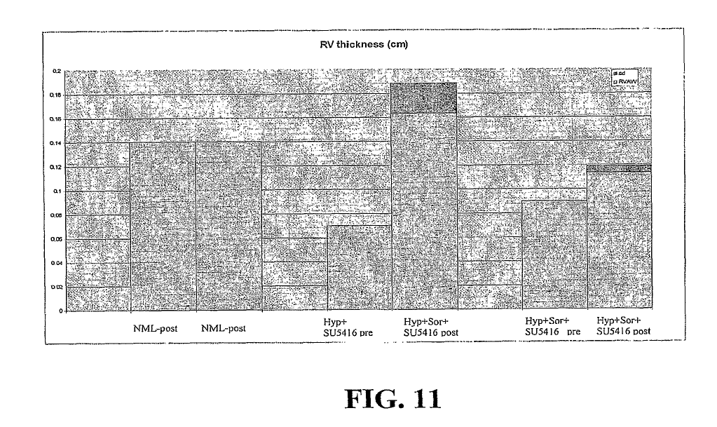

FIG. 14 Shows a time course of clinical status, exercise, and hemodynamic before and after initiation of imatinib treatment. The figure demonstrates the improvement in functional class 6MW distance and decrease in pulmonary vascular resistance (patient had a decrease in both mean PAP and an increase in cardiac index). (Ghofrani et al., 2005)

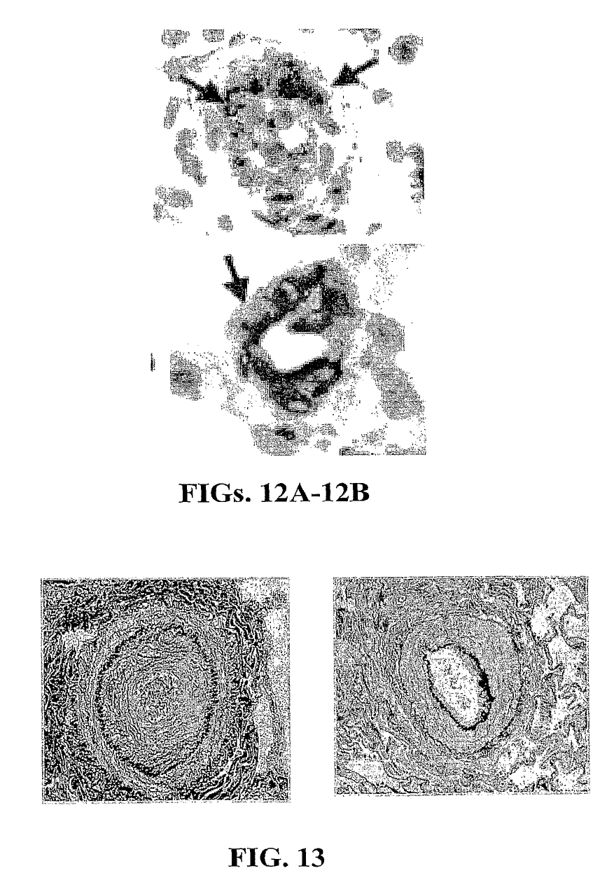

FIGS. 15A-15B Representative photomicrographs of rat lung at 21 days. Hematoxylin+Eosin stained (H&E) sections (10×). A. Hypoxia+SU5416 and B. Hypoxia+SU5416+Sorafenib.

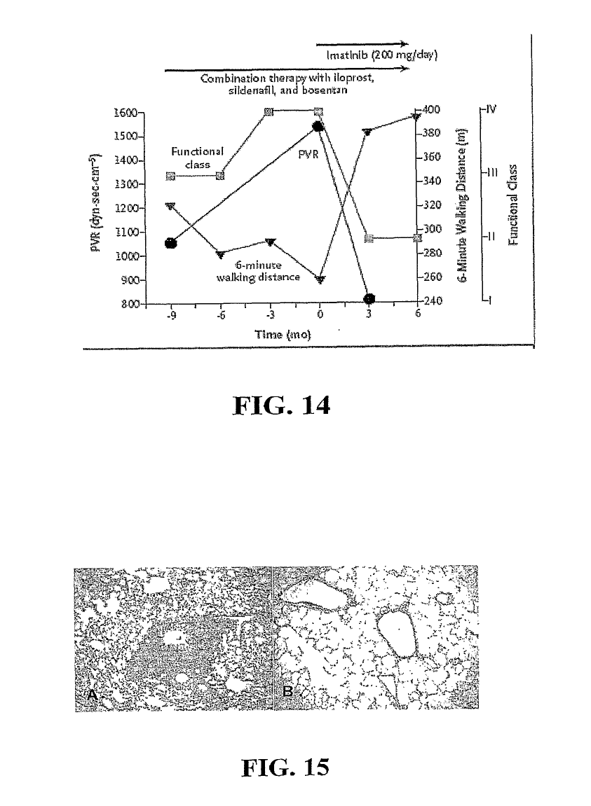

FIG. 16 Shows histopathology of rat lung under normoxia, Hypoxia, Hypoxia+SU5416+Sorafenib, Hypoxia+Sorafenib, and Hypoxia+SU5416. The sections show remodeling of the lung vasculature in injured animals (Hypoxia+SU5416) and a dramatic improvement in rats that received daily sorafenib (Hypoxia+SU5416+Sorafenib), almost to Normoxia controls

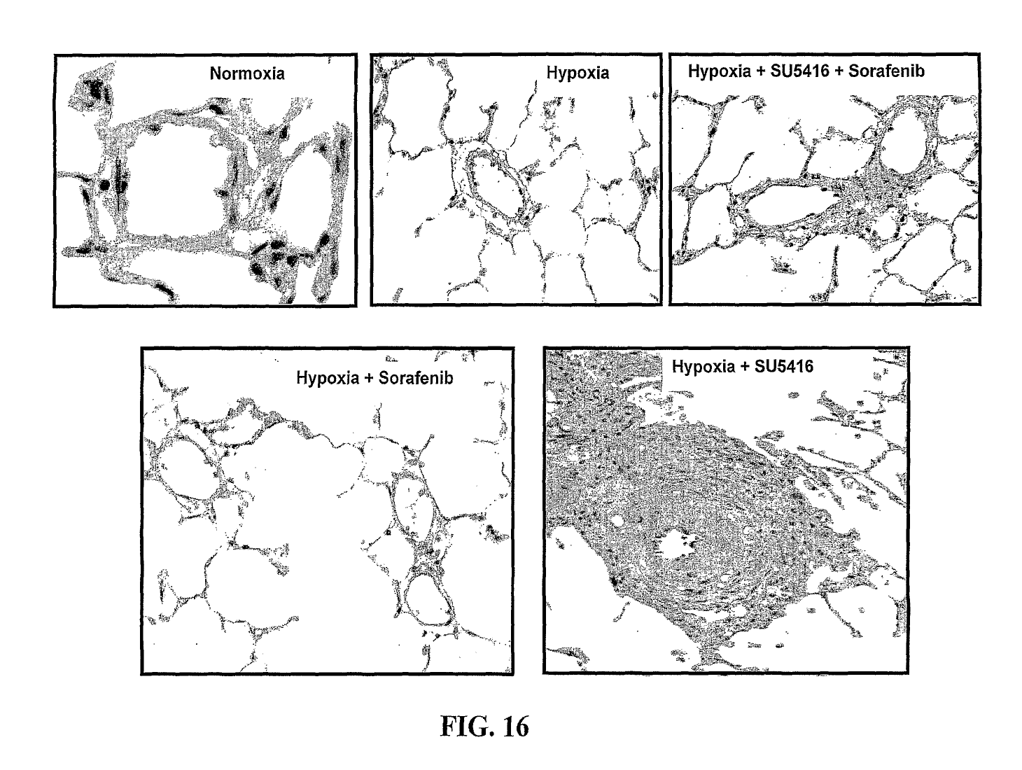

FIG. 17 Shows the Heath-Edwards grading of animals under normoxia, Hypoxia, Hypoxia+SU5416+Soarfenib, Hypoxia+Sorafenib, and Hypoxia+SU5416. The Heath-Edwards grading system grades pulmonary hypertension by microscopic features. The general classification is as follows:

Potentially Reversible—grade I Hypertrophy of the media of muscular pulmonary arteries. Extension of muscle into the wall of pulmonary arterioles; grade II Muscle hypertrophy plus proliferation of intimal cells in arterioles and small muscular arteries; grade III Muscle hypertrophy plus subendothelial fibrosis. Eventually, concentric masses of fibrous tissue and reduplicated internal elastic lamina occlude the vascular lumen of arterioles and small muscular arteries. Large elastic arteries show atherosclerosis

Usually Irreversible—grade IV Muscle hypertrophy is less apparent; progressive dilatation of small arteries, especially those near vessels with intimal fibrous occlusion. Plexiform lesions occur; grade V Plexiform and angiomatoid lesions plus intra-alveolar hemosiderin-filled macrophages; grade VI Necrotizing arteritis with thrombosis. Fibrinoid necrosis of the arterial wall with a transmural infiltrate of polymorphonuclear leukocytes and eosinophils.

### DETAILED DESCRIPTION OF THE INVENTION

Pulmonary arterial hypertension is an angiogenic proliferative vasculopathy resulting from abnormal endothelial and smooth muscle cell interactions. Over time, the vasculopathy causes a narrowing of the pulmonary artery and its branches, resulting in right heart failure and death. Therapies are directed primarily at dilating the narrowing vessels, and include: prostacyclins (epoprostenol, treprostinil, and iloprost), endothelin receptor blockers (bosentan, sitaxsentan and ambrisarten, which is in the FDA approval process), and phosphodiesterase inhibitors (sildenafil and tadalafil (investigational). All currently FDA-approved therapies improve six minute walk distance, (average 30 meters) with minimal change in hemodynamic measurements and only epoprostenol has a proved survival benefit. Drugs that inhibit processes important to pathological blood vessel branching and growth, represent a new class of therapeutic agents for pulmonary arterial hypertension.

Sorafenib is a bi-aryl urea that inhibits Raf-1 kinase, a regulator of endothelial apoptosis, VEGFR-2, PDGFR-β, and VEGFR-3, growth factor receptors necessary for angiogenesis, affecting vascular permeability and vessel stabilization through pericyte recruitment and maturation. The inventors have designed and executed a three week study to assess the safety and therapeutic activity of sorafenib in a hypoxia-induced model of pulmonary hypertension in Dahl SS rats. A compound with a reportedly more limited spectrum of kinase inhibitory activity, SU5416, was previously demonstrated to exacerbate pulmonary hypertension in hypoxic rats.

Pulmonary hypertension refers to elevated blood pressure in the pulmonary circulation. Pulmonary hypertension can be either primary or secondary to pulmonary or cardiac disease. Typically, the pulmonary blood pressure in humans suffering from pulmonary hypertension is greater than a mean pulmonary artery pressure of 25 mm Hg. The common symptoms of PAH include dyspnea, fatigue, weakness, chest pain, recurrent syncope, seizures, light-headedness, neurologic deficits, leg edema and palpitations (Rich, 1987; The Merck Manual, 1999).

One embodiment of the invention encompasses methods of treating, pallating, preventing, and/or managing PAH by administration to a patient in need of such treatment, prevention, or management a therapeutically or prophylactically effective amount of sorafenib, or a pharmaceutically acceptable derivative or prodrug thereof.

As used herein, and unless otherwise indicated, the terms “pulmonary arterial hypertension,” “PAH” and pulmonary hypertension “PH” and related disorders include, but are not limited to: Idiopathic PAH; Familial PAH, and Associated PAH, which is related to certain conditions including connective tissue disease, congenital systemic-to-pulmonary-shunts, portal hypertension, HIV infection, drugs and toxins, glycogen storage disease, Gaucher's disease, hereditary hemorrhagic telangiectasia, hemoglobinopathies, myeloproliferative disorders, splenectomy, and others; PAH associated with significant venous or capillary involvement; and persistent pulmonary hypertension of the newborn. It is contemplated that embodiments discussed in the context of PAH may be applied in the context of PH, and vice versa.

The term “prodrug” as used in this application refers to a precursor or derivative form of a pharmaceutically active substance that is capable of being enzymatically or non-enzymatically activated or converted into the more active parent form. See, e.g., Wilman (1986) and Stella et al. (1985). In certain aspects, a prodrug is less cytotoxic, has a longer half-life, may be targeted to a particular tissue and/or is more stable than the parent drug. For example, an amide group of sorafenib can be derivatized to a hydroxyalkyl or acycloxymethyl derivative or other chemically appropriate derivative. The hydroxyalkyl may then be converted non-enzymatically to render sorafenib. The acycloxymethyl group can be acted upon by an esterase, which is then followed by a non-enzymatic conversion to sorafenib.

Another embodiment of the invention encompasses a method of treating, preventing and/or managing PH, which comprises administering or prescribing to a patient in need of such treatment, prevention and/or management a therapeutically or prophylactically effective amount of sorafenib, or a pharmaceutically acceptable salt or prodrug thereof, and a therapeutically or prophylactically effective amount of a second agent.

Examples of second agents include, but are not limited to, anticoagulants, diuretics, cardiac glycosides, calcium channel blockers, vasodilators, prostacyclin analogues, endothelin antagonists, phosphodiesterase inhibitors, endopeptidase inhibitors, lipid lowering agents, thromboxane inhibitors, or other agents found, for example, in the Physician's Desk Reference 2003. Second agents can be large molecules (e.g., proteins) or small molecules (e.g., synthetic inorganic, organometallic, or organic molecules). Examples of specific second agents include, but are not limited to, amlodipine, diltiazem, nifedipine, adenosine, epoprostenol (Flolan®), treprostinil (Remodulin®), bosentan (Tracleer®), warfarin, digoxin, nitric oxide, L-arginine, iloprost, betaprost, and sildenafil (Viagra®).

Another embodiment of the invention encompasses a method of reversing, reducing or avoiding an adverse effect associated with the administration of a therapeutic used to treat PH, which comprises administeringor prescribing to a patient in need thereof a therapeutically or prophylactically effective amount of sorafenib, or a pharmaceutically acceptable salt or prodrug thereof, and an optional second agent.

Procedures such as lung transplantation may be necessary to treat PH patients who have failed to respond to medical therapy. It is believed that the combined use of sorafenib and lung transplantation in a patient suffering from PH can be beneficial. It is believed that sorafenib can work in combination with transplantation therapy, reducing complications such as chronic rejection and opportunistic infections associated with the transplantation. Therefore, this invention encompasses a method of treating or managing PH, which comprises administering to a patient (e.g., a human) sorafenib, or a pharmaceutically acceptable salt or prodrug thereof, before, during, or after transplantation therapy.

Certain aspects of the invention encompass pharmaceutical compositions that can be used in methods of the invention. Specific compositions comprise sorafenib, or a pharmaceutically acceptable salt or prodrug thereof, and an optional second agent.

Also encompassed by the invention are single unit dosage forms of sorafenib, or a pharmaceutically acceptable salt or prodrug thereof.

Other aspects of the invention can encompass kits which comprise sorafenib, or a pharmaceutically acceptable salt or prodrug thereof, and a second agent. For example, a kit may contain the compound of the invention, and calcium channel blockers, vasodilators, prostacyclin analogues, endothelin antagonists, phosphodiesterase inhibitors, endopeptidase inhibitors, lipid lowering agents, thromboxane inhibitors or other agents used to treat PAH patients.

As part of the spectrum of sorafenib's pharmacologic activity overlaps with SU5416, a study was performed to determine whether sorafenib had similar effects or with additional mechanisms of inhibitory activity could counteract the effects of SU5416. Dahl Salt Sensitive rats were divided into 5 groups: normoxia+vehicle (normal, healthy control), hypoxia+SU5416 (positive control), hypoxia+sorafenib (safety comparison with SU5416), and hypoxia+sorafenib+SU5416 (therapeutic activity assessment). Echocardiograms were performed on all rats at the start of the study. Except for the normoxia group, all rats were maintained in a hypoxia chamber with a partial pressure of oxygen of 10% FiO₂ for the duration of the study. Rats in the two SU5416 groups received one subcutaneous injection of SU5416 at the start of the study (20 mg/kg). Stock sorafenib solutions were prepared every three days, crushing and dissolving sorafenib tablets in EL/ethanol (50:50; Sigma Cremophor EL, 95% ethyl alcohol) at final concentration of 4 mg/mL, protected from light exposure and stored at room temperature. Final dosing solutions were prepared on the day of use by dilution of the stock solution to 1 mg/mL with water and administered by gavage to the rats daily. After 3 weeks, all rats had echocardiography and hemodynamic studies. Organ and blood specimens were obtained for further evaluation. Echocardiography images were obtained with short axis views demonstrating RV wall thickness when comparing Hypoxia+SU5416+sorafenib; Hypoxia+SU5416; and Control-Normoxia. RV 3DE measurements were also determined (diameters+area), A=RV major diameter, B=RV annulus diastolic diameter & area, C=RV at ⅓ RV major diameter & area, D=RV at ⅔ RV major diameter & area.

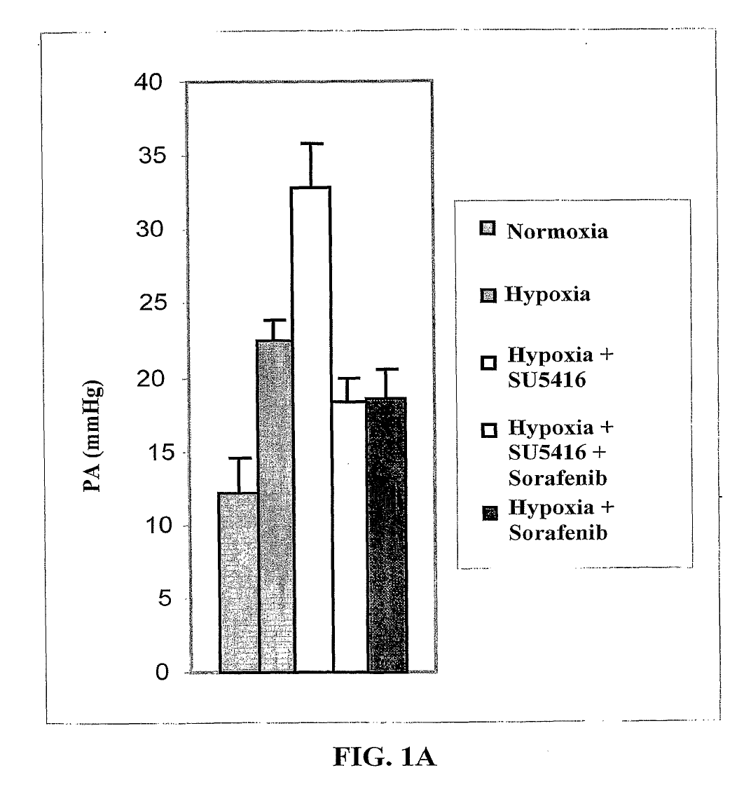

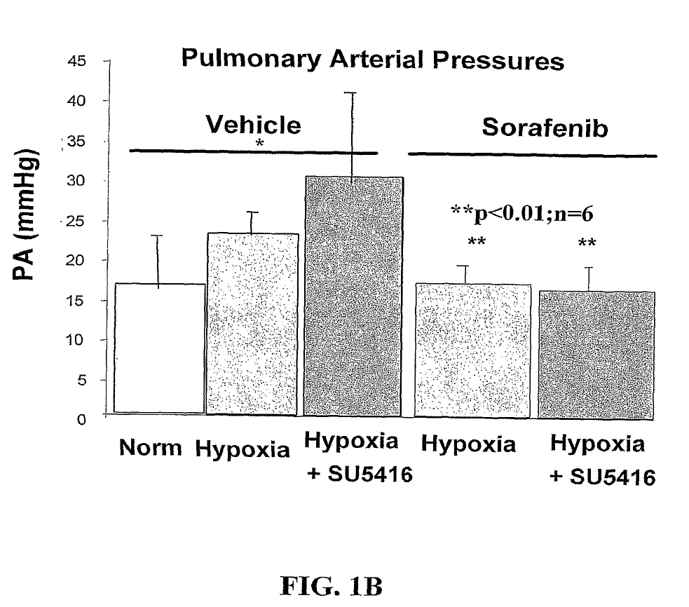

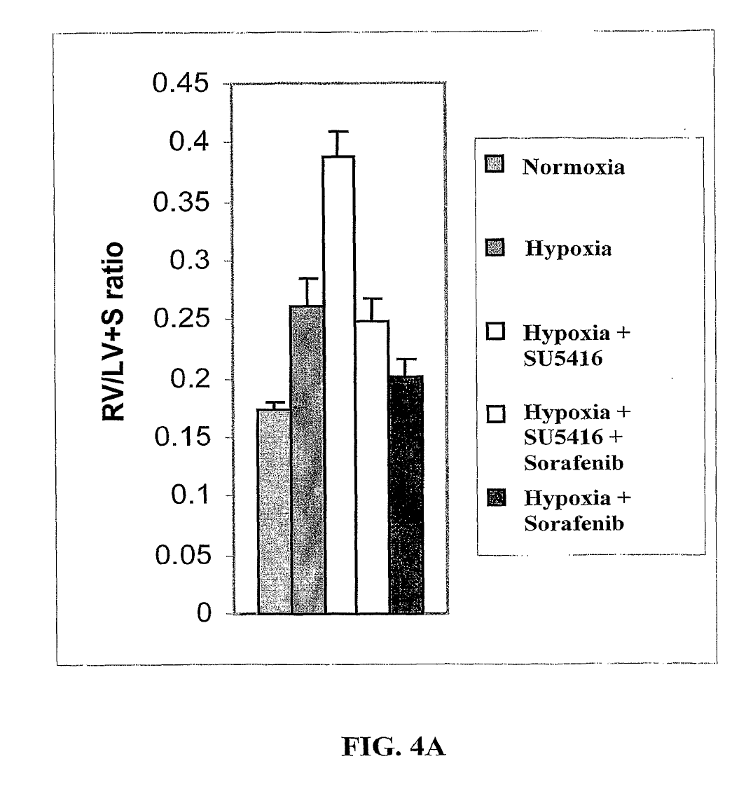

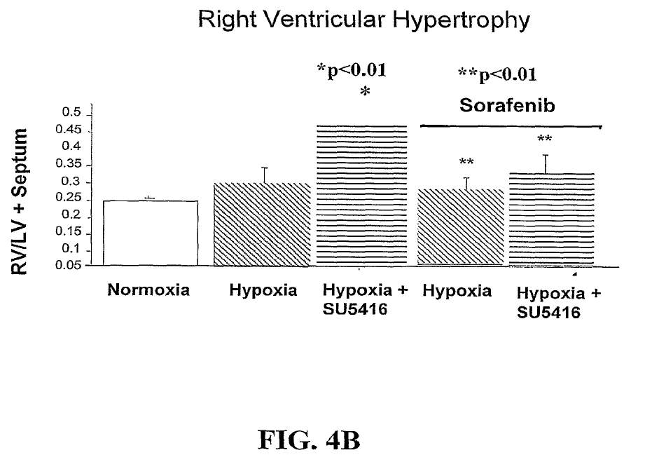

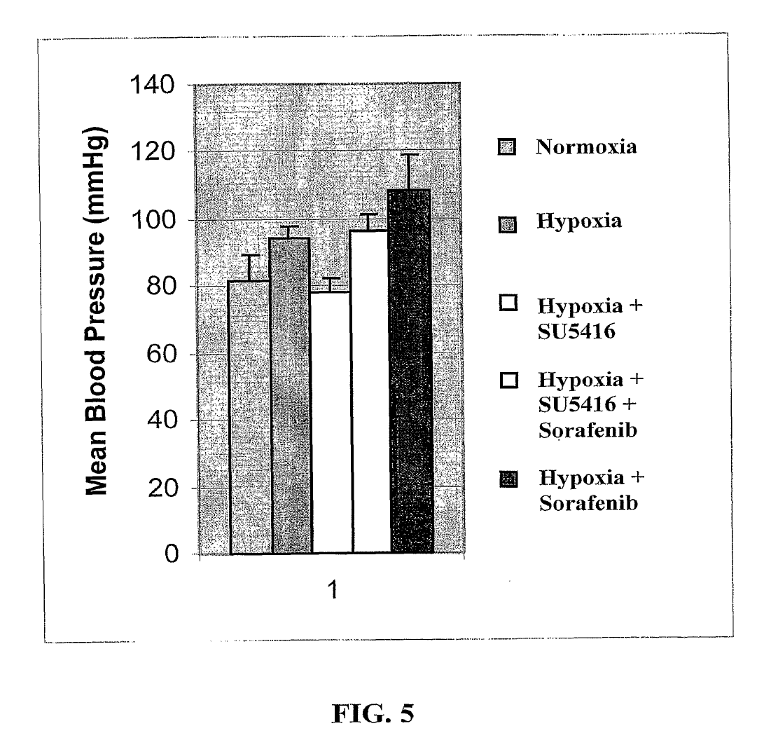

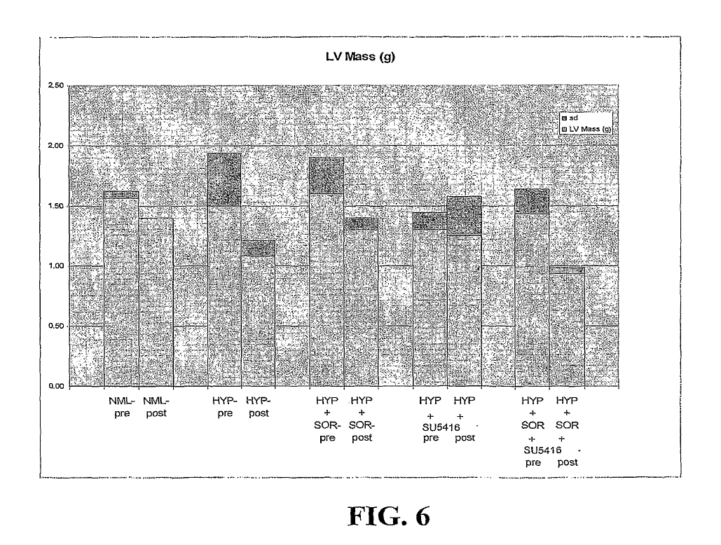

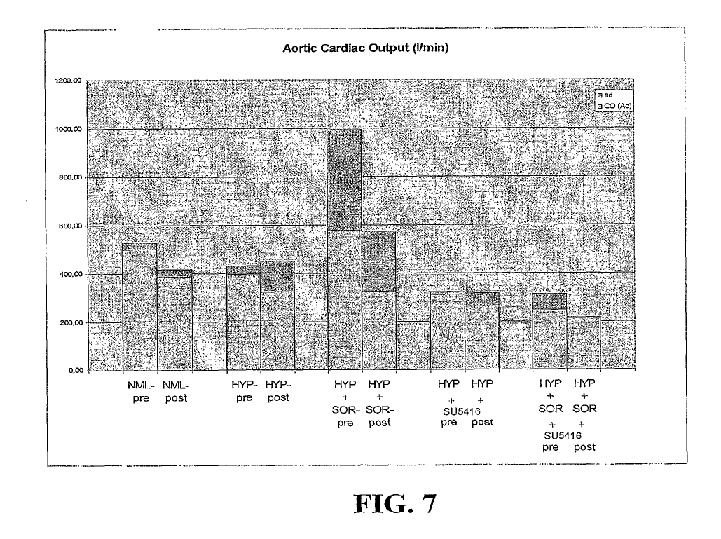

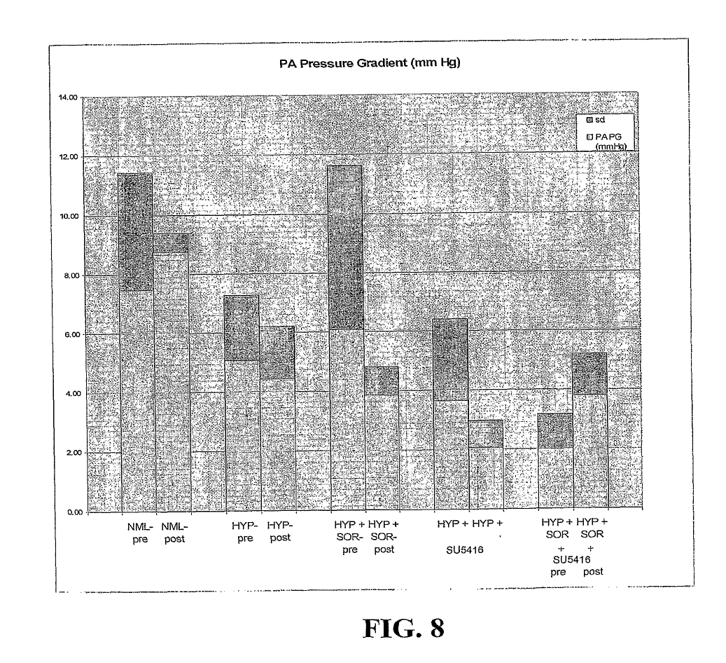

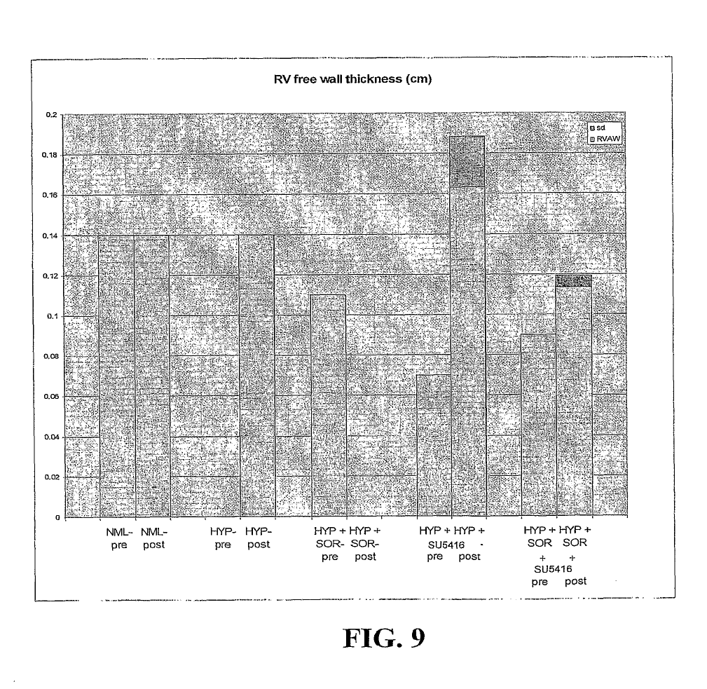

As has been previously described, rats given SU5416 developed pulmonary hypertension measured by elevated right ventricular and pulmonary artery pressures, echocardiographic changes, and elevated right ventricle/left ventricular weights. Rats exposed to hypoxia had mildly elevated pressures compared with normoxia and there was no significant change in pressures or weights in rats given hypoxia plus sorafenib. Sorafenib appears to have a beneficial effect on pulmonary hypertensive rats as rats in hypoxia, plus SU5416, plus sorafenib had pressures and weights similar to normoxia. The small sample size did not allow for significant change in echocardiographic data but there appeared to be a positive trend with this combination. Pathologic specimen results are pending. Based on these results the experiments were repeated with 18 rats distributed in the same 5 groups (only difference was 3 additional rats in the normoxia group control group). The results were reproducible. In the initial experiment two rats died during induction of anesthesia for echocardiographic analysis (after unblinding-normoxia, hypoxia/SU5416). In the subsequent experiment, one rat died on day zero (after unblinding-1 hypoxia/sorafenib on day three). These data suggest that sorafenib is a safe, therapeutic agent for the treatment of human pulmonary hypertension. Below are the results of the 2 experiments as one cohort. Also see FIGS. 1-11.

TABLE 1
Pressures derived from SU5416 + sorafenib study - 3½ Wk
Hypoxia + Sorafenib (Daily 2.5 mg/Kg) SU5416 (day zero 20 mg/kg i.p.)

| | PA mmHg | RVSP mmHg | RV/LV kg (+/−) | BP mmHg | |
|---|---|---|---|---|---|
| | (+/−) SE | (+/−) SE | SE | (+/−) SE | N |
| Normoxia Vehicle | 12 +/− 2 | 11 +/− 3 | 0.173 +/− 0.007 | 82 +/− 8 | 7 |
| Hypoxia | 22 +/− 1 | 20 +/− 2 | 0.262 +/− 0.023 | 94 +/− 4 | 6 |
| Hypoxia + SU5416 | 33 +/− 3 | 30 +/− 4 | 0.389 +/− 0.02 | 78 +/− 4 | 5 |
| Hypoxia + Sorafenib | 18 +/− 2 | 17 +/− 2 | 0.248 +/− 0.02 | 97 +/− 4 | 6 |
| Hypoxia + Sorafenib + | 19 +/− 2 | 17 +/− 3 | 0.202 +/− 0.015 | 108 +/− 10 | 5 |
| SU5416 | | | | | |

Sorafenib appears to protect SU5416-treated hypoxia-exposed rats from pulmonary arterial hypertension. Furthermore, while sorafenib and SU5416 share some pharmacological activity, unlike SU5416 sorafenib does not exacerbate hypoxia-induced hypertension. These data suggest that sorafenib is safe and a potential therapeutic agent for the treatment of human pulmonary hypertension.

III. Compounds of the Invention

Sorafenib is a small molecule inhibitor of Raf kinase and Raf associated signaling pathways, including extracellular regulated kinases (ERKs). Sorafenib is also known as BAY 43-9006, its anti-cancer properties are described in US Patent Publication 20030125359, which is incorporated herein by reference. Sorafenib is known to target both the Raf/MEK/ERK signaling pathway inhibiting cell proliferation and the VEGFR-2/PDGFR-β pathway inhibiting angiogenesis. Sorafenib is currently being tested in the United States by Bayer/Onyx in phase III clinical trials for advanced renal cell carcinoma. Methods for the preparation of sorafenib and its related compounds is described in U.S. Patent Publication number 2001/0027202 and 2003/0139605, which are incorporated herein by reference in their entirety.

A. Tyrosine Kinase/VEGFR Inhibitors

A number of anti-cancer therapeutics are classified as tyrosine kinase inhibitors (for a review see Levitski and Mishani, 2006, which is incorporated herein by reference in its entirety). Tyrosine kinases include a number of cell surface receptors such as the VEGF receptors. VEGF is one of the key regulators for both physiological and pathological angiogenesis. Because of the multitude of cellular responses that are initiated and regulated by VEGF, and because of its specificity for the vascular endothelium, VEGF takes an exceptional position among other growth factors (Petrova et al. Exp Cell Res, 253:117-130, 1999).

There are three VEGF-receptors (VEGFR1, VEGFR2, and VEGFR3). VEGFR1 is mainly expressed in hematopoietic stem cells, macrophages and monocytes as well as in vascular endothelium. VEGFR2 is more characteristic of the vascular and lymphatic endothelium, whereas VEGFR3 is predominantly expressed in lymphatic endothelium (Cross et al., 2003).

Since VEGFR2 is generally considered the most important transducer of VEGF-dependent angiogenesis, this receptor represents a major target within the angiogenesis-related kinases. VEGFR2 inhibitors include, but are not limited to abt-869, amg706, AZD2171, bay57-9352, bay43-9006 (sorafenib), XL647, XL999, GW786034, bevacizumab, PKC412, AEE788, PTK787 (vatalanib), OSI-930, OSI-817, SU11248, AG-013736, ZK3-4709, quinazoline ZD6474, pyrrolocarbazole CEP-7055 (orally active N,Ndimethylglycine ester of CEP-5241), and CP-547632. VEGFR2 inhibitors are generally kliowii or can be identified using VEGFR2 kinase assays.

A. Second Agents

Sorafenib can be combined with other pharmacologically active compounds (“second agents”) in methods and compositions of the invention. In a preferred embodiment, the second agents are capable of reducing pulmonary artery pressure or vascular resistance, inhibiting thrombosis or thromboembolism, or ensuring compliance of patients. Examples of the second agents include, but are not limited to, anticoagulants, diuretics, cardiac glycosides, calcium channel blockers, vasodilators, prostacyclin and prostacyclin analogues, endothelin receptor antagonists, phosphodiesterase inhibitors (e.g., PDE 5 inhibitors), endopeptidase inhibitors, lipid lowering agents, thromboxane inhibitors, and other therapeutics known to reduce pulmonary artery pressure.

Specific second agents are anticoagulants, which are useful in the treatment of patients with PH who have an increased risk of thrombosis and thromboembolism. A particular anticoagulant is warfarin (Coumadin®).

Other second agents include diuretics, cardiac glycosides, and oxygen. Digoxin therapy is used to improve right ventricular function in patients with right ventricular failure. Diuretics can be used to manage peripheral edema. Oxygen supplementation may be used in those patients with resting or exercise-induced hypoxemia.

Calcium channel blockers such as diltiazem, amlodipine, and nifedipine can also be used as second agents, particularly for vasoreactive patients at right heart catheterization. These drugs are thought to act on the vascular smooth muscle to dilate the pulmonary resistance vessels and lower the pulmonary artery pressure (Tapson, 2002).

Other second agents include vasodilators, particularly for NYHA/WHO class III and IV patients with right heart failure who do not respond to calcium channel blockers or are unable to tolerate them. Examples of vasodilators include, but are not limited to, prostacyclin (e.g., prostaglandin I₂ (PGI₂), epoprostenol (EPO, Flolan®), treprostinil (Remodulin®)), and nitric oxide (NO).

Still other second agents are endothelin antagonists. One example is bosentan (Tracleer®), which competitively binds to endothelin-1 (ET-1) receptors A+B, causing reduction in pulmonary artery pressure.

Specific second agents used in the invention include, but are not limited to, amlodipine, nifedipine, diltiazem, bosentan (Tracleer®), prostacyclin (e.g., epoprostenol (Flolan®), treprostinil (Remodulin®), iloprost), warfarin (Coumadin®), tadalafil (Clalis®), simvastatin (Zocor®), omapatrilat (Vanlev®), irbesartan (Avapro®), pravastatin (Pravachol®), digoxin, nitric oxide, L-arginine, iloprost, betaprost, and sildenafil (Viagra®).

IV. Methods of Treatment and Management

A. Diagnosis

Pulmonary Hypertension is typically defined as a pulmonary artery mean pressure greater than 20 mm Hg with a pulmonary vascular resistance greater than two Wood units. Pulmonary hypertension is indicated by increased shortness of breath during exertion accompanied by one of the known causes of PH. Methods of diagnosing PH include echocardiography, Doppler flow studies, assessment of blood oxygenation, pulomonary function, computer tomography of the chest, ventilation-perfusion lung scanning, and cardiac catheterization (Nauser and Stites, 2001). Pulmonary hypertension left untreated results in right ventricular failure and death. Diagnosis of PH may be associated with identification of right ventricular hypertrophy on an ECG or prominent pulmonary arteries on a chest radiograph. Once an indication of PH has been establish a patient will typically undergo two-dimensional echocardiography with Doppler flow studies. Typically, PH is confirmed by identification of tricuspid regurgitation with right ventricular enlargement and/or dysfunction.

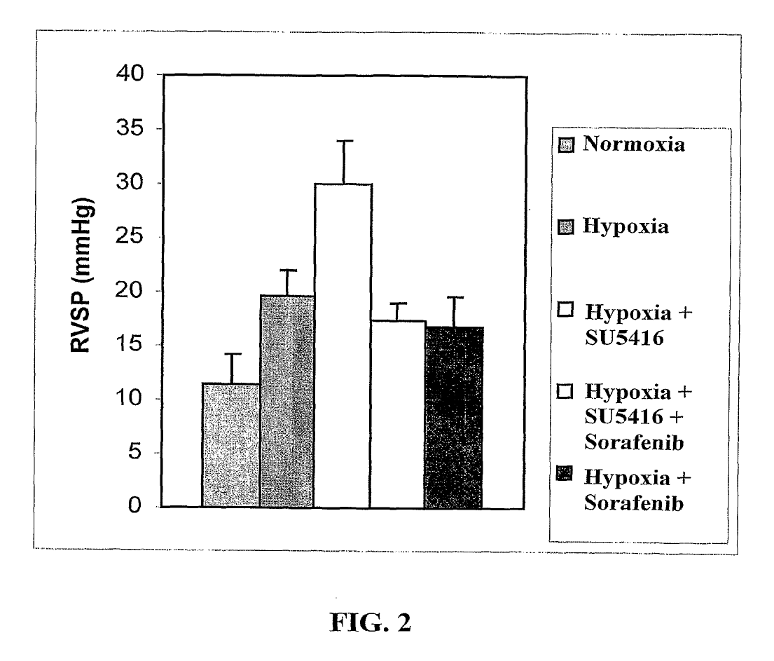

Once a patient has been diagnosed with PH, the patient should undergo testing to identify any underlying causes for PH. The test include, but are not limited to blood analysis, including blood count, prothrombin time, partial thromboplastin time, hepatic profile, autoimmune panel, basic naturetic peptide (BNP), and HIV testing; blood gas analysis; pulmonary function testing; CT scan; ventilation-perfusion lung scan; cardiac catheterization; or combinations thereof (see Nauser and Stites, 2001 figure 3 algorithm for evaluation of a patient with suspected pulmonary hypertension).

The familial and medical history of a patient may be used to identify a subject at risk of developing PH and is a candidate for prophylactic or preventative treatments.

B. Treatment

Treatment of PH depends on the stage and the mechanism of the disease. Typical treatments for PH include, but are not limited to correction of underlying cause (e.g., surgical treatment of mitral stenosis, left to right shunt, or accessible chronic thromboemboli; afterload reduction, digoxin and diuretics for left ventricular dysfunction; prevention and treatment of respiratory infection; avoidance of anorectic agents); decrease pulmonary vascular resistance (e.g., vasodilation (oxygen, calcium channel blockers, prostacyclins, nitric oxide), or anticoagulation); increase cardiac output (short-term parenteral inotropes or digoxin); reduce volume overload (low-salt diet or diuretics); or surgery (lung transplant or atrial septosotomy) (see Nauser and Stites, 2001 table 5 possible treatments for pulmonary hypertension).

Several studies suggest that survival is increased when the patient is treated with anticoagulant therapy, regardless of histopathologic subtype of PAH (Rubin et al., 1997). Warfarin is used to maintain an International Normalized Ratio of 1.5- to 2-times the control value, provided no contraindication to anticoagulation is present (Tapson, 2002). Warfarin is the standard of care for thromboembolic PH.

Digoxin is used to prevent and treat supraventricular arrhythmias associated with PAH and for patients who have concomitant left heart failure. However, no randomized controlled clinical study has been performed to validate this strategy for patients with IPAH (Tapson, 2002). Diuretics are reportedly useful in reducing excessive preload in patients with right heart failure (Rubin et al., 1997). Oxygen supplementation is used in those patients with resting or exercise-induced hypoxemia (Rubin et al., 1997; Tapson, 2002).

Arterial septostomy or lung transplant is indicated for patients who do not respond to medical therapy (The Merck Manual, 1999; Rubin, 2002). Arterial septostomy is intended to serve as a bridge to transplantation.

Medications presently used for the treatment of PH include calcium channel blockers and pulmonary vasodilators (The Merck Manual, 1999; Tapson, 2002). Calcium channel blockers are utilized for “true responders” as evidenced by response during right heart catheterization (McLaughlin et al., 2004, which is incorporated herein by reference in its entirety). Vasodilators include, but are not limited to epoprostenol, treprostinil, and iloprost. Endothelin antagonists include, but are not limited to Tracleer and the like. Phosphodiesterase inhibitors include, but are not limited to sildanefil and the like.

Methods of this invention encompass methods of preventing, treating and/or managing various types of PH, particularly PAH. As used herein, unless otherwise specified, the term “preventing” or “prophylaxis” includes, but is not limited to, inhibiting or averting one or more symptoms associated with PH. Symptoms associated with PH include, but are not limited to, dyspnea, fatigue, weakness, chest pain, recurrent syncope, seizures, light-headedness, leg edema, and palpitations. As used herein, unless otherwise specified, the term “treating” refers to the administration of a composition after the onset of symptoms of PH, whereas “preventing” refers to the administration prior to the onset of symptoms, particularly to patients at risk of PH. As used herein and unless otherwise indicated, the term “managing” encompasses preventing the recurrence of PH in a patient who had suffered from PH, and/or lengthening the time that a patient who had suffered from PH remains in remission.

The invention encompasses methods of treating or managing patients who have been previously treated for PH, as well as those who have not previously been treated for PH. Because patients with PH have heterogenous clinical manifestations and varying clinical outcomes, it is preferred that patients should be treated according to the severity and stage of the disease. Methods and compositions of this invention can be used in various stages or types of PH including, but not limited to, primary PH, secondary PH and WHO classes I to IV patients.

Methods encompassed by this invention comprise administering sorafenib, or a pharmaceutically acceptable salt or prodrug thereof to a patient (e.g., a human) suffering, or likely to suffer, from PH. In one aspect of the invention, sorafenib is administered in single or divided daily doses in an amount of from about 0.1 to about 1000 mg/day, from about 100 to about 800 mg/day, or from about 50 to about 400 mg/day.

C. Combination Therapy

Administration of sorafenib and a second agent to a patient can occur simultaneously or sequentially by the same or different routes of administration. The suitability of a particular route of administration employed for a particular agent will depend on the agent itself (e.g., whether it can be administered orally without decomposing prior to entering the blood stream) and the disease being treated. A preferred route of administration for sorafenib is oral. Another preferred route of administration for sorafenib is parenteral, particularly for patients who are in a peri-transplant period or in an end stage of PH. Preferred routes of administration for the second agent of the invention are known to those of ordinary skill in the art such as in Physicians' Desk Reference (2003).

Various combinations may be employed, sorafenib is “A” and a second agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the present invention to a patient will follow general protocols for the administration of agents for the treatment of PAH. It is expected that the treatment cycles would be repeated as necessary. It also is contemplated that various standard therapies, as well as surgical intervention, may be applied in combination with the described compositions and formulations.

The specific amount of the second agent will depend on the specific agent used, the type of PH being treated or managed, the severity and stage of PH, and the amount(s) of sorafenib and any optional additional agents concurrently administered to the patient. In specific embodiments of the invention, the second agent is amlodipine, diltiazem, nifedipine, prostacyclins (e.g., epoprostenol (Flolan®), treprostinil (Remodulin®), iloprost), bosentan (Tracleer®), warfarin (Coumadin®), tadalafil (Clalis®), simvastatin (Zocor®), omapatrilat (Vanlev®), irbesartan (Avapro®), pravastatin (Pravachol®), digoxin, nitric oxide, L-arginine, beraprost, or sildenafil (Viagra®).

In one embodiment of the invention, sorafenib is administered to reduce a period of treatment with a second agent typically used to treat PH. In a particular embodiment, at the beginning of week one, from about 100 to about 800 mg/day of sorafenib is administered along with a second agent in an amount that those of ordinary skill in the art can determine by their professional judgment. At the beginning of weeks 5, 9, 13, and 17, withdrawal of the second agent may occur in increments of 25% of the initial dose of the second agent. At the beginning of week 17, dose of the second agent may be 800 mg/day if symptoms of a patient do not worsen. If symptoms of a patient worsen, dose of the second agent may be increased to stabilize the patient.

D. Combination with Surgery or Transplantation

This invention encompasses a method of treating or managing PH, which comprises administering sorafenib, or a pharmaceutically acceptable salt or prodrug thereof, in conjunction with surgery or transplantation therapy. As discussed herein, the treatment of PH varies, depending on the stage and mechanism of the disease. Arterial septostomy or lung transplantation may be necessary for PH patients who have failed to respond to other therapy. Sorafenib may provide additive or synergistic effects when given before, concurrently with, or after surgery or transplantation therapy in patients with PH.

V. Pharmaceuticals and Methods for the Treatment of Disease

In additional embodiments, the present invention concerns formulation of sorafenib compositions disclosed herein in pharmaceutically-acceptable solutions for administration to a cell, tissue, animal, or patient either alone, or in combination with one or more second agent or second therapy.

Aqueous pharmaceutical compositions of the present invention will have an effective amount of a sorafenib that modulates PH and/or its related pathologies or etiologies. Such compositions generally will be dissolved or dispersed in a pharmaceutically acceptable carrier or aqueous medium. An “effective amount,” for the purposes of therapy, is defined at that amount that causes a clinically measurable difference in the condition of the subject. This amount will vary depending on the substance, the condition of the patient, the type of treatment, etc.

The phrases “pharmaceutically” or “pharmacologically acceptable” refer to molecular entities and compositions that do not produce a significant adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredients, its use in the therapeutic compositions is contemplated. Supplementary active ingredients, such as other anti-diabetic agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, inhalants and the like.

The active compounds of the present invention will often be formulated for parenteral administration, e.g., formulated for injection via the intravenous, intramuscular, subcutaneous, or even intraperitoneal routes. The preparation of a composition that contains sorafenib alone or in combination with a second therapeutic agent as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In many cases, the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts include the acid addition salts and which are formed with inorganic acids such as, for example, hydrochloric or phosphoric acids, or such organic acids as acetic, oxalic, tartaric, mandelic, and the like. Salts formed can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 1035-1038 and 1570-1580). Some variation in dosage will necessarily occur depending on the condition of the subject being treated. The person responsible for administration will, in any event, determine the appropriate dose for the individual subject.

In certain aspects of the methods of the invention, the route that the therapeutic composition is administered may be by parenteral administration. The parenteral administration may be intravenous injection, subcutaneous injection, intramuscular injection, ingestion or a combination thereof. In certain aspects, the composition comprising sorafenib is administered from about 0.1 to about 10 microgram/kg/body weight per dose. In certain aspects, the composition comprising sorafenib is administered from about 1 to about 5 microgram/kg/body weight per dose. In certain aspects, the composition comprising sorafenib is administered from about 1.2 to about 2.4 microgram/kg/body weight per dose. In certain aspects, the amount of sorafenib administered per dose may be about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9. about 3.0, about 3.1, about 3.2, about 3.3, about 3.4, about 3 5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8. about 9.9 to about 10.0 or more nanogram/kg/body, microgram/kg/body, or milligram/kg/body.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

A. Alimentary Delivery

The term “alimentary delivery” refers to the administration, directly or otherwise, to a portion of the alimentary canal of a subject or patient. The term “alimentary canal” refers to the tubular passage that functions in the digestion and absorption of food and the elimination of food residue, which runs from the mouth to the anus, and any and all of its portions or segments, e.g., the oral cavity, the esophagus, the stomach, the small and large intestines and the colon, as well as compound portions thereof such as, e.g., the gastro-intestinal tract. Thus, the term “alimentary delivery” encompasses several routes of administration including, but not limited to, oral, rectal, endoscopic and sublingual/buccal administration. A common requirement for these modes of administration is absorption over some portion or all of the alimentary tract and a need for efficient mucosal penetration of the agent so administered.

1. Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup or elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

2. Endoscopic Administration

Endoscopy can be used for therapeutic delivery directly to an interior portion of the alimentary tract. For example, endoscopic retrograde cystopancreatography (ERCP) takes advantage of extended gastroscopy and permits selective access to the biliary tract and the pancreatic duct (Hirahata et al., 1992). However, the procedure is unpleasant for the patient, and requires a highly skilled staff.

3. Rectal Administration

Therapeutics administered by the oral route can often be alternatively administered by the lower enteral route, i.e., through the anal portal into the rectum or lower intestine. Rectal suppositories, retention enemas or rectal catheters can be used for this purpose and may be preferred when patient compliance might a otherwise be difficult to achieve (e.g., in pediatric and geriatric applications, or when the patient is vomiting or unconscious). Rectal administration may result in more prompt and higher blood levels than the oral route, but the converse may be true as well (Remington's Pharmaceutical Sciences, 711, 1990). Because about 50% of the therapeutic that is absorbed from the rectum will bypass the liver, administration by this route significantly reduces the potential for first-pass metabolism (Benet et al., 1996).

B. Injectable Delivery

In certain circumstances it will be desirable to deliver the pharmaceutical compositions disclosed herein parenterally, intravenously, intramuscularly, or even intraperitoneally as described in U.S. Pat. Nos. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that it is easy to use a syringe.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration (see for example, Remington's Pharmaceutical Sciences, 1035-1038 and 1570-1580. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

4. Parenteral Delivery

The term “parenteral delivery” refers to the administration of a therapeutic of the invention to an animal in a manner other than through the digestive canal. Means of preparing and administering parenteral pharmaceutical compositions are known in the art (see, e.g., Remington's Pharmaceutical Sciences, pages 1545-1569, 1990).

5. Intraluminal Administration

Intraluminal administration, for the direct delivery of a therapeutic to an isolated portion of a tubular organ or tissue (e.g., such as an artery, vein, ureter or urethra), may be desired for the treatment of patients with diseases or conditions afflicting the lumen of such organs or tissues. To effect this mode of administration, a catheter or cannula is surgically introduced by appropriate means. After isolation of a portion of the tubular organ or tissue for which treatment is sought, a composition comprising a therapeutic of the invention is infused through the cannula or catheter into the isolated segment. After incubation for from about 1 to about 120 minutes, during which the therapeutic is taken up or in contact with the cells of the interior lumen of the vessel, the infusion cannula or catheter is removed and flow within the tubular organ or tissue is restored by removal of the ligatures which effected the isolation of a segment thereof (Morishita et al., 1993). Therapeutic compositions of the invention may also be combined with a biocompatible matrix, such as a hydrogel material, and applied directly to vascular tissue in vivo.

C. Nasal Delivery

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

D. Epidermal and Transdermal Delivery

Epidermal and Transdermal Delivery, in which pharmaceutical compositions containing therapeutics are applied topically, can be used to administer drugs to be absorbed by the local dermis or for further penetration and absorption by underlying tissues, respectively. Means of preparing and administering medications topically are known in the art (see, e.g., Remington's Pharmaceutical Sciences, 1596-1609, 1990).

E. Liposome-, Nanocapsule-, and Microparticle-Mediated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the nucleic acids or constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Chonn, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. Nos. 5,567,434; 5,552,157; 5,565,213; 5,738,868 and 5,795,587, each specifically incorporated herein by reference in its entirety).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 [mgr]m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandary et al., 1995 and U.S. Pat. No. 5,145,684, specifically incorporated herein by reference in its entirety).

VI. Kits

In some cases, agents of the invention are not administered to a patient at the same time or by the same route of administration. This invention therefore encompasses kits which, when used by the medical practitioner, can simplify the administration of appropriate amounts of agents to a patient.

A typical kit or composition of the invention comprises a dosage form of sorafenib, or a pharmaceutically acceptable salt or prodrug thereof. Kits encompassed by this invention can further comprise additional active agents such as amlodipine, dilitazem, nifedipine, adenosine, epoprostenol (Flolan®), treprostinil (Remodulin®), bosentan (Tracleer®), warfarin (Coumadin®), tadalafil (Clalis®), simvastatin (Zocor®), omapatrilat (Vanlev®), irbesartan (Avapro®), pravastatin (Pravachol®), digoxin, nitric oxide, L-arginine, iloprost, beraprost, and sildenafil (Viagra®), or a combination thereof. Examples of the additional active agents include, but are not limited to, those disclosed herein.

Kits of the invention can further comprise devices that are used to administer the active agents. Examples of such devices include, but are not limited to, syringes, drip bags, patches, and inhalers.

Kits of the invention can further comprise pharmaceutically acceptable vehicles that can be used to administer one or more active agents. For example, if an active agent is provided in a solid form that must be reconstituted for parenteral administration, the kit can comprise a sealed container of a suitable vehicle in which the active agent can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Pulmonary Hypertension Connection Database/Pulmonary Hypertension Specimen Collection

The Pulmonary Hypertension Connection (PHC) has collected baseline demographic, clinical phenotype, medication, echocardiography, exercise test, and cardiac catheterization data for 900 patients followed by the inventors practice since its inception in 1980. Development of this database began at Rush and has continued at the University of Chicago. Currently established at the University of Chicago, the Pulmonary Hypertension Program has over 1200 active patients. Since receiving IRB-approval over 1,000 active patients have provided informed consent and a full-time data manager continues to input data on past and active patients. The PHC stores clinical data but also provides statistical analysis software in the single unit for rapid analysis by a trained investigator.

In January 2005, the inventors' protocol to generate a longitudinal specimen collection entitled, “Biomarkers In Pulmonary Hypertension” received IRB approval. The protocol is for collection of DNA, peripheral blood specimens, and clinically indicated central blood specimens. Discovery of relevant biomarkers in these specimens can be immediately and safely integrated into a prospective study and routine clinical care. Patients evaluated by pulmonary hypertension specialists who consent to the collection and archiving of plasma, serum, and DNA are currently enrolled. Blood is collected during clinically indicated evaluations either from a peripheral vein at scheduled office visits or from central catheters placed during cardiac catheterization. Plasma, serum, and DNA are isolated, frozen and stored by standard techniques in accord with International Society for Biological and Environmental Repositories (ISGER) best practices (Somiari et al., 2004; Friede et al., 2003). Coding of these specimens allows for retrospective selection of appropriate specimens to test potential biomarkers for prediction/association with specific clinical phenotypes identified in the PHC.

### Example 2

### Development of Novel Endpoints

The clinical assessment of patients with pulmonary hypertension currently involves an assessment of exercise capacity, echocardiography, and cardiac hemodynamics. Overall assessment is based on a combination of these factors but there have not been any guidelines on treatment based on the overall assessment, only based on worsening functional class and cardiac hemodynamics. Invasive hemodynamics is still the most reliable and most accurate assessment of pulmonary vascular resistance. Catheterization does not provide information on the patient's functional ability, right ventricular or pulmonary vascular reserve, or on pathologic vascular remodeling of the pulmonary circulation (regression, identification of plexiform lesions). Alternative non-invasive techniques may prove to be better biomarkers for PAH. The following experimental endpoints will be evaluated as novel measures of disease activity: Naughton-Balke treadmill test (TT), 3DE, and 64 slice CT scan.

Naughton-Balke Treadmill Test (TT)-6MW is preformed by the subject walking on a flat surface at his/her own pace. The 6MW is not a true measure of exercise from a physiology perspective. Cardiopulmonary exercise testing in few centers is a better correlate of exercise physiology and hemodynamics, (Oudiz et al., 2006; Yasunobu et al., 2005) but this test is time consuming, expensive, and difficult to do with reproducibility in unexperienced centers. The University of Chicago center is gaining expertise in this non-invasive test and may in the future incorporate this testing in protocols. The metabolic equivalent, or MET, is the amount of oxygen used by a seated person and is a predictor of survival in the general population. It is a physiologic measure of exercise capacity. The Naughton-Balke exercise protocol, (Patterson et al., 1972) measured in METs is easy to administer by programming a standard treadmill, is easily reproducible, inexpensive, and has been preformed by programs at the University of Chicago for many years. Because of the inventors familiarity with this test, and because it is a better measure of exercise physiology than the 6MW, it is contemplated that the MET will be a better predictor and measure to follow for PH patients.

Using data from the Intravenous Treprostinil, study I determined the reliability of MET compared to the 6MW. Both tests were done by each patient in the study at multiple time points. Pearson correlation coefficient (r) was calculated to express inter-relationship between measures of exercise capacity. Intra-class correlation coefficient (ICC) was used to indicate the repeated reliability of exercise capacity measures over time. 6MW and MET were symmetrically distributed and correlated (r=0.63). ICCs for 6MW, TT, and MET were 0.77, 0.82, and 0.80, respectively. Functional class correlated with 6MW and MET at baseline and at wk 12 (all p<0.01), but hemodynamics did not correlate with exercise measures consistently. Based on this result, MET is as reliable and reproducible as the 6MW. The next step is to prove that MET is a better predictor and surrogate endpoint than the 6MW.

Three-Dimensional Echocardiography (3DE)—DE software (TomTec, Germany) has been developed that semi-automatically detects RV endocardial borders and addresses drawbacks of prior RV quantification. Patients have RT3DE as part of their routine evaluation with two-dimensional echocardiography (2DE). Investigation of the influence of different degrees of pulmonary hypertension on RV remodeling in patients and development of novel indices that may better assess this phenomenon are under way. Using a matrix array probe and Sonos 7500 (Philips, Mass.), a wide-angled acquisition of the RV during a breath hold gated to ECG will be obtained. The RV volume will be analyzed using software that semi-automatically detects RV endocardial borders resulting in global and regional RV volume and ejection fraction (EF). Several RV indices will include RV diameter in 2D and 3D, 3D area measurements at the tricuspid valve (TV) annulus, ⅓ of the RV and ⅔ of the RV.

In initial observations 3D echocardiographers at the University of Chicago, found that RV remodeling is most evident at ⅓ of the RV. The TV annulus does not significantly dilate until patients have severe PH. The inventors anticipate that this technology will expand the knowledge of how the RV reacts towards changes in pressure, volume overload or different disease states. Patients will continue to have RT3DE in Phase I/II trials to assess and monitor response to therapy.

Computed Tomography (CT) Scans—At the University of Chicago under the direction of a leading radiologist in computer based analysis of radiographic imaging; the inventors have developed a fully automated lung segmentation method for thoracic CT scans that has been used for automated analysis of lung parenchyma texture, including the lung texture of patients with PH. A preliminary study of 28 CT scans (14 from PH patients and 14 from aged-matched “normal” patients) was conducted. The lung texture analysis method was applied to a single section from each CT scan; these individual sections were manually selected at the level of the bifurcation of the pulmonary artery. The lung texture features were used to discriminate between the normal and the PH groups. This discrimination task was performed by a linear discriminant classifier, the performance of which was evaluated by receiver operating characteristic (ROC) analysis with the LABROC4 program (C. E. Metz, Ph.D, The University of Chicago). The automated method achieved an area under the ROC curve of 0.97 (out of a maximum 1.0) in this discrimination task (submitted ERS). Based on this preliminary study, all patients seen by our PH center are asked to enroll in the CT database—a repository allowing high level analyses of scans. Shortly all PH patients with clinical indications for a chest CT will have a 64 slice CT to continue work on this project and to determine the ability to detect plexiform lesions.

### Example 3

Preclinical Data with Dahl-Sensitive Rats

Human Pulmonary Hypertension (PH) is an often fatal vascular disorder affecting ˜2-3 new cases/million/yr characterized by increased pulmonary artery pressure, right heart failure, and death. Current strategies to treat PH are problematic and do not address the vascular remodeling (smooth muscle contraction, hypertrophy) characteristic of the disease. PH exhibits significant overlap with cancer pathophysiology with abnormalities in signal transduction and cellular proliferation (endothelium, smooth muscle) resulting in an angioproliferative vasculopathy. As cancer and pulmonary hypertension share the involvement of angiogenesis and growth factor pathways, it was hypothesized that sorafenib may represent a novel therapeutic agent for PAH. The inventors set out to conduct pre-clinical studies to evaluate the safety and efficacy of sorafenib in a rodent model of hypoxia-induced PAH. This rat model combines hypoxia and SU5416, a VEGFR-2 inhibitor, which together produce PH characterized by: vasoconstriction, elevated pulmonary artery pressure, right ventricular hypertrophy, and vascular remodeling. (Taraseviciene-Stewart et al. FASEB J. 2001; 15:427-438). Another feature of this model is smooth muscle medial thickening and endothelial cell apoptosis.

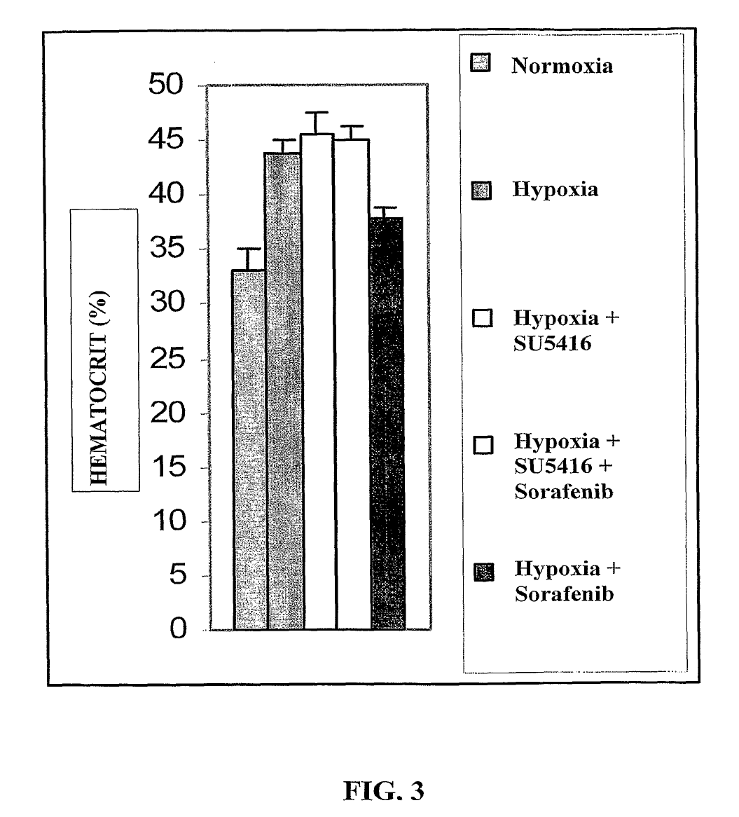

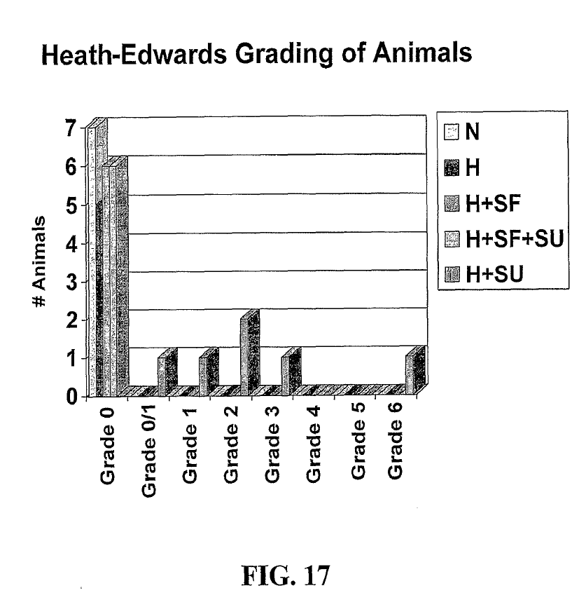

Protocol includes an initial administration of SU5416 subcutaneously at 20 mg/kg followed daily by sorenfenib oral daily doses of 2.5 mg/kg. Some animals were exposed to chronic hypoxia (10% O₂). The animals were divided into five experimental groups: hypoxia+sorafenib; hypoxia+SU5416+sorafenib, normoxia+vehicle control; Hypoxia+vehicle; and hypoxia+SU5416. PAP, RVSP, Echo, and DNA microarrays were used to measure various physiological and molecular biological parameters. FIG. 4 illustrates that hypoxia/SU5416-induces increases in right heart pressures and right heart hypertrophy (RV/LV+Septum). FIG. 4 also illustrates that sorafenib prevented hypoxia+SU5416 development of right heart hypertrophy (RV/LV+Septum) in Dahl SS rats. FIG. 1B illustrates that sorafenib prevents hypoxia+SU5416 induced pulmonary hypertension and remodeling. Sorafenib Prevents Remodeling in Dahl SS with PH (Health Edwards Grading) H/E sections (FIG. 17).

To study potential mechanisms of sorafenib effects on PH RNA was isolated from rat lung tissue and analyzed using the Rat Chip II (Affymetrix). Sorafenib attenuated physiologic and histopathologic changes in PAH in a rodent model of PH. Mechanisms of PAH pathobiology is poorly understood therefore, to explore potential mechanistic pathways the inventors conducted bioinformatic studies and expression profiling. Expression profiling revealed at least 179 genes differentially regulated by sorafenib, when comparing profiling data to normoxia using GCRMA normalization in R and SAM (δ>0.639, minimum fold change>1.7). The candidate genes identified fell into various gene ontology classes, for example 4% were in the lung development and growth factors class (e.g., Bambi, Tgfb3, Ltbp2), 13% in the cell migration/ECM class (e.g., Itga3_predicted, Cspg4, Cxcll2, Reln, Col18a1, Cthrc1, Hmgcr, Cnn1, Col6a2, Jag2), 4% in the apoptosis class (e.g., Anzal, Col18a1, Jag2), 4% in the smooth muscle/fiber formation class (e.g., Des, Tpm3, Jph2), 10% in the cell proliferation class (e.g., Anxal, Timp1, Lamb_{—}1 predicted, Cspg4, Cxcll2, Gja4, Jag2, Tgfb3), 6% in the blood vessel development class (e.g., Cxcll2, Cspg4, Gja4, Col18a1, Serpine1), and 5% in the angiogenesis class (Cxcl12, Cspg4, Col18a1, Serpine1).

TABLE 2
Gene ontology for sorafenib influenced gene, genes known to be involved in PH.

| | | | PubMatrix Terms | | |
|---|---|---|---|---|---|
| Gene | | Pulmonary | Vascular | | |
| Symbol | Gene Name | Hypertension | Remodeling | Hypoxia | Endothelium |
| DES | desmin | 103 | 33 | 308 | 585 |
| Mcpt10 | Mast cell protease 10 | 4 | 3 | 2 | 13 |
| Timp1 | Tissue inhibitor of | 11 | 71 | 30 | 118 |
| | metalloprotease 1 | | | | |
| Fbn1 | Fibrillin 1 | 2 | 5 | 0 | 18 |
| Nupr1 | Nuclear protein 1 | 104 | 140 | 1680 | 1646 |
| Tgfb3 | Transforming growth, | 42 | 111 | 102 | 442 |
| | factor beta 3 | | | | |
| Bmpr1a | Bone morphogenetic | 8 | 2 | 1 | 4 |
| | protein receptor, type | | | | |
| | 1A | | | | |
| Bmpr2 | Bone morphogenetic | 121 | 10 | 7 | 18 |
| | protein receptor, type II | | | | |
| Itga6 | Integrin, alpha 6 | 2 | 11 | 17 | 364 |
| Trpc6 | Transient receptor | 5 | 3 | 3 | 9 |
| | potential cation channel, | | | | |
| | subfamily C member | | | | |

TABLE 3
Gene ontology for sorafenib influenced genes, unknown genes

| | | PubMatrix Terms | | | |
|---|---|---|---|---|---|
| | Unknown genes | Pulmonary | Vascular | | |
| Gene symbol | Gene name | hypertension | remodeling | Hypoxia | Endothelium |
| Ankrd1 | Ankyrin repeat domain 1 | 0 | 0 | 3 | 4 |
| Cnn1 | Calponin1 | 0 | 7 | 2 | 22 |
| Chst12 | Carbohydrate | 0 | 0 | 0 | 1 |
| | sulfotransferase 12 | | | | |
| Hmgcs1 | 3-hydroxy-3- | 0 | 0 | 0 | 1 |
| | methylglutaryl- | | | | |
| | coenzyme A synthase 1 | | | | |
| Cyp51 | Cytochrome P450, | 0 | 0 | 0 | 2 |
| | subfamily 51 | | | | |
| Esm1 | Endothelial cell-specific | 0 | 1 | 4 | 53 |
| | molecule 1 | | | | |
| Gja4 | Gap junction membrane | 0 | 0 | 0 | 1 |
| | channel protein alpha 4 | | | | |
| Cxcl12 | Chemokine (C-X-C | 0 | 14 | 26 | 149 |
| | motif) ligand 12 | | | | |
| Jam3 | Junctional adhesion | 0 | 0 | 1 | 14 |
| | molecule 3 | | | | |
| Anxa1 | Annexin A1 | 0 | 1 | 2 | 27 |
| Ltbp2 | Latent transforming | 0 | 2 | 1 | 12 |
| | growth factor beta | | | | |
| | binding protein 2 | | | | |
| Mgl1 | Macrophage galactose | 0 | 0 | 0 | 2 |
| | N-acetyl-galactosamine | | | | |
| | specific lectin 1 | | | | |

The inventors have successfully utilized sorafenib to attenuate (physiologic and histopathologic) the development of rodent pulmonary hypertension model (Dahl SS) utilizing chronic hypoxia and SU5416 administration. Gene expression profiling studies identified genes which are well recognized to be involved in angiogenesis, endothelial cell apoptosis, and PH (Table 2), as well as genes previously not associated with PH representing potential novel candidate genes (Table 3). Given its safety in advanced cancer patients, further studies exploring both the mechanism of action as well as human studies, evaluating sorafenib safety and efficacy in PH should be pursued.

Preclinical data with Dahl-sensitive rats given sorefenib with hypoxia and SU5416 did not develop evidence of PAH. Sorafenib may have a beneficial effect in the treatment PAH. The PHC database enables the phenotype our patients to be determined based on clinical data, which will be a useful tool in discriminating response to therapy. Non-invasive methods that may better evaluate and prove to be better screening and biomarkers in PAH include exercise treadmill testing by a Naughton-Balke protocol, 3DE, and CT scanning.

### Example 4

### Clinical Studies

The pathophysiology of PAH overlaps the pathophysiology of cancer with aberrancies in signal transduction leading to abnormal endothelial and smooth muscle cell interactions and to angioproliferative vasculopathy. New signal transduction inhibitors being evaluated for the treatment of cancer represent potential effective therapies for PAH. With an existing phase I cancer therapeutics program, the University of Chicago provides a unique environment to improve PAH care. Having established that the recently FDA-approved agent, sorafenib, an inhibitor of multiple kinases important to angiogenesis (Raf-1 kinase inhibitor, VEGFR-2, VEGFR-3, PDGFR-β), protected rats from developing PAH in the SU5416/hypoxia model of PAH, the inventors contemplate evaluation of this agent in Phase I and Phase II trials.

A. Single Center Phase IB Trial of Sorafenib in PAH Patients.

Study Objectives Phase IB study: To determine the tolerability of oral daily sorafenib in combination with prostacyclin+sildenafil in pulmonary hypertension patients. This design is a single-center, Phase IB dose escalation study of sorafenib up to the known MTD of 400 mg twice daily. Administration will continue until the occurrence of unacceptable toxicity (described below), withdrawn consent, disease progression, hospitalization for PH/right heart failure, lung transplantation, or death.

Accrual: The PH program is currently following over 150 subjects on stable prostacyclin therapy. Phase I will enroll 12 subjects and be completed within 6-9 months

Rationale for Inclusion Criteria: Prostacyclin therapy is the most efficacious therapy currently available for PAH. Long-term prostacyclin replacement is supported by the pathophysiology of PAH, the relative lack of prostacyclin seen in PAI, and prostacyclin's positive effects on pulmonary vascular bed. However, prostacyclin therapy is not curative. As a class, the side effect profile of all prostacyclins is similar to that of epoprostenol and is usually minimal and well tolerated in most subjects. These include flushing, headache, nausea, loose stool, jaw discomfort with “first bite”, and foot pain with prolonged standing or walking (Barst et al., 1996; McLaughlin et al., 2002; Sitbon et al., 2002).

Epoprostenol, the first approved therapy for PAH, has a half-life of no more than 1-2 minutes, mandating continuous intravenous therapy via a central catheter (Data et al., 1981). Cohort analyses in Europe and in the United States have provided convincing evidence of its long term benefits (McLaughlin et al., 2002; Sitbon et al., 2002). Because of its pharmacology, prostacyclin analogues were developed to ease administration. Treprostinil is a tricyclic benzidene prostacyclin analogue that shares pharmacologic actions with epoprostenol (Clapp et al., 2002) Treprostinil differs from epoprostenol in that it is chemically stable at room temperature and neutral pH and has a longer half-life (3-4 hours) (Wade et al., 2004). Subcutanous treprostinil was approved in 2002 for the treatment of New York Heart Association (NYHA) class II-IV PAH patients (Simonneau et al., 2002) and recently, the FDA approved the use of intravenous treprostinil based on bioequivalence to subcutaneous therapy (Laliberte et al., 2004). Iloprost is a chemically stable prostacyclin analog that can be delivered by inhaler/nebulizer (Hoeper et al., 2000). This allowed a targeted approach, with direct inhalation of prostacyclin for more selective pulmonary effects. But because of its short duration of action it must be inhaled 6-12 times daily (Olschewski et al., 2002; Hoeper et al., 2000).

To enhance prostacyclin benefits, combination therapeutic approaches and inhibiting multiple pathways concurrently may produce additive benefit. Others have examined combining a prostacyclin with agents that increase cyclic guanasine phosphate (cGMP). This is accomplished by inhibition of the phosphodiesterase 5 enzyme (PDE-5) which degrades cGMP in the vascular smooth muscle cell. The pulmonary vasculature has a higher concentration of the PDE-5 enzyme than most vascular beds. Oral sildenafil in combination with iloprost in (Hoeper et al., 2000) PAH patients over 9 to 12 months follow-up improved exercise capacity and hemodynamics (Ghofrani et al., 2003). Open uncontrolled experience adding sildenafil to epoprostenol also improved hemodynamics (Stiebellehner et al., 2003) and open label addition to subcutaneous treprostinil improved exercise capacity (Gomberg-Maitland et al., 2005). Large multicenter trials are currently in progress. Of note, animal data obtained prior to the large scale epoprostenol plus sildenafil study consisted of safety and efficacy of sildenafil not the drugs in combination. There is a reported animal study with beraprost (oral formulation not approved in U.S) plus sildenafil using the monocrotaline rat model that did not demonstrate any safety/toxicity and demonstrated efficacy and improved survival (Itoh et al., 2004). Previous sorafenib studies have not demonstrated known drug interactions with warfarin or digoxin. Patients on other classes of PAH therapy (endothelin receptor antagonists, arginine), or experimental therapies will not be included based on the lack of conclusive data and on the preference to examine the drug in a homogenous patient population.

PAH is a devastating disease. Ethically, for Phase I/II trials based on current available therapeutic agents, the inventors believe that all subjects should have significant exercise capacity limitation based on the 6MW and be receiving prostacyclin therapy with or without sildenafil.

Patient Selection-Eligibility Criteria:

1. Age>18 years

2. PAH as defined as IPAH, FPAH or PAH associated with connective tissue disease. (Humbert et al., 2004)

3. Baseline 6MW>150 meters and <450 meters

4. PAH as defined by hemodynamics at diagnosis by right heart catheterization defined as: mean PAP>25 mmHg with a normal PCWP≦15 mm Hg at rest and a PVR>2 Wood units

5. Receiving conventional therapy as clinically indicated (oxygen, diuretics, aldosterone antagonist, calcium channel blockers, digoxin) with dose that is unchanged in the preceding 30 days prior to enrollment. This is excluding anticoagulants (warfarin) as the patient's dose may not be stable if the patient is having a cardiac catheterization at baseline within 30 days of enrollment and warfarin is being held. The dose of warfarin needs to be stable for 7 days or therapeutic with an INR=2.0

6. On intravenous/subcutaneous prostacyclin at a stable dose >30 days

7. Subjects must be on sildenafil at a stable dose >30 days.

8. Must have right heart catheterization on prostacyclin+sildenafil within preceding 30 days. Subjects must be on a stable dose of medication within 30 days prior to cardiac catheterization and therefore there can be no dosage changes of the medications between catheterization and baseline.

9. Must have pulmonary function tests (PFT) within 90 days prior to enrollment: TLC, FEV1, FVC, DLCO

10. Women of childbearing years must use adequate contraception (hormonal or barrier method of birth control) prior to enrollment

11. Ability to understand and the willingness to sign a written informed consent document

Exclusion Criteria:

1. PAH associated with all other etiologies: HIV, portopulmonary disease, congenital heart disease (Humbert et al., 2004)

2. Subjects with pulmonary hypertension due to thromboembolism, significant interstitial lung disease, chronic obstructive pulmonary disease, congestive heart failure, valvular heart disease (Humbert et al., 2004)

3. Subjects with (World Health Organization (WHO) functional Class IV (Hiumbert et al., 2004)

4. Subjects with scleroderma with total lung capacity (TLC)<60% of predicted within 30 days of screening

5. Subjects with significant obstructive lung disease with FEV1<80% of predicted

6. Subjects with hypotension defined as systolic arterial pressure <90 mmHg at baseline

7. Subjects with hypertension defined as systolic arterial pressure >140 mmHg at baseline and a diastolic arterial pressure >90 mmHg.

8. Subjects with impaired renal function as defined as creatinine clearance <30 ml/min as defined by the Cockcroft-Gault formula: Male: Creatitine clearance (ml/min)=(140-age)×(body weight in kg)/(72× serum creatinine in mg/dl); Female: Creatitine clearance (ml/min)=0.85 (140-age)×(body weight in kg)/(72× serum creatinine in mg/dl)

9. Subjects with liver function tests (transaminases (AST/ALT), total bilirubin, and alkaline phosphatase)>2× normal values

10. Subjects with acutely decompensated heart failure or hospitalization within the previous 30 days prior to screening

11. Subjects may not be receiving any other investigational agents

12. Subjects on endothelin receptor antagonists (bosentan, sitaxsentan, ambrisentan) or chronic arginine supplementation

13. Subjects with left ventricular ejection fraction <45% or left ventricular shortening fraction <0.2

14. Subjects with acute myocardial infarction within 90 days prior to screening

15. Subjects with limitations to performance of exercise measures (6MW) due to conditions other than PH associated dyspnea/fatigue

16. Subjects taking nitrates for any medical problem

17. Subjects taking phosphodiesterase inhibitors (any formulation) for erectile dysfunction

18. Subjects with a recent (<180 days) history of pulmonary embolism verified by ventilation/perfusion scan, angiogram or spiral CT scan

19. Pregnant or lactating women

20. Subjects with a history of current drug abuse including alcohol

Treatment Plan:

Each subject will be individually dose escalated to a maximum of 400 mg twice daily. The starting dose of sorafenib will be 200 mg daily. If tolerated at the completion of 1 month, the dose will be increased to 200 mg twice daily. If dose-limiting toxicity (DLT) occurs, dose escalation will be terminated and that dose level will be denoted as the maximum administered dose (MAD) for this subject. The subject will continue on the dose preceding the last escalation to complete a total of 4 months of active therapy. If this dose is not tolerated the subject will be withdrawn. If 200 mg twice daily is tolerated at the conclusion of month 2, the dose will be increased to 400 mg twice daily. If 400 mg twice daily is tolerated at the end of month 3 it will be continued until the completion of month 4.

Dose Limiting Toxicity (DLT) is defined as intolerable prostacyclin side effects: flushing, headache, nausea, loose stool, jaw discomfort with “first bite”, and foot pain with prolonged standing or walking. Intolerable side effects from phosphodiesterase inhibitors: headache, gastrointestinal distress, hypotension. Intolerable side effects from sorafenib: rash, diarrhea, fatigue, hypertension, hand-foot syndrome.

Grading of DLT will occur on a 3 point scale for prostacyclin and phosphodiesterase side effects: 1=stable, 2=increased but tolerable, 3=increased+intolerable. Prostacyclin dose will not be down-titrated for any reason in this investigational trial. If this is required the subject will be withdrawn.

Grading of DLT for sorafenib will be based on the Common Terminology for Adverse Event oncology grading scale 1-5. (CTAE V3.0-Dec. 12, 2003). The three most common adverse events include diarrhea and hand-foot skin reaction with the following grading: Diarrhea: 1=increase of <4 stools per day over baseline, 2=increase of 4-6 stools per day over baseline; IV fluids indicated <24 hours, 3=increase of >7 stools per day over baseline; incontinence; IV fluids >24 hours; hospitalization, 4=life threatening consequences, and 5=death. Hand-foot skin reaction: 1=minimal skin changes or dermatitis (e.g. erythema) without pain, 2=skin changes (e.g. peeling, blisters, bleeding, edema) or pain, not interfering with function, and 3=ulcerative dermatitis or skin changes with pain interfering with function. Hypertension: 1=asymptomatic, transient (<24 hours) increase by >20 mmg (diastolic) or to 150/100 mmHg if previously normal; intervention not indicated, 2=recurrent or persistent (>24 hours) or symptomatic increase by >20 mmHg (diastolic) or to >150/100 mmHg if previously normal; monotherapy may be indicated, 3=requiring more than one drug or more intensive therapy than previously, 4=Life threatening consequences (e.g. hypertensive crisis), 5=death. Subjects will be withdrawn with CTAB grade 3 diarrhea, hand-foot skin reaction, or hypertension.

DLT Cardiovascular/PH toxicity is defined as arrhythmia, worsening right heart failure, hospitalization for worsening right heart failure/PH, worsening dyspnea, worsening WHO class, worsening exercise capacity as defined as a decrease in 6MW>20% from baseline or a decrease of >30 meters with subjective or clinical signs and symptoms of progression.

Expected Adverse Events (AE): Prostacyclin side effects: include flushing, headache, nausea, loose stool, jaw discomfort with “first bite”, and foot pain with prolonged standing or walking. Sildenafil side effects: gastrointestinal discomfort, headache, flushing. Sorafenib side effects: rash, diarrhea, fatigue, hypertension, hand-foot syndrome.

Withdrawal: Subjects withdrawn from protocol due to clinical deterioration or DLT will have an office visit 30-40 days after end of treatment to record the following: physical exam, blood pressure, PH symptoms, WHO class, concomitant medications/adverse events, dose of prostacyclin, 6MW and TT.

Data Safety Monitoring: Weekly meeting by the PH research team will discuss all subjects enrolled for data safety monitoring.

Data Collection: Subjects will have a screening visit prior to enrollment including a history and physical exam. At this visit subjects will have two-6MW with Borg Dyspnea Score (B)>2 hrs apart up to 1 day later, as per ATS guidelines, using phrases of standard encouragement (ATS statement, 2002). The second test will be used for screening purposes and to limit variability. The mean reported increase ranges from 0 to 17% preformed a day later (ATS statement, 2002). Alternatively this can be done on separate days within 14 days. All patients will have an assessment of WHO functional class, and if needed PFT and a right heart catheterization as stated by the protocol. WHO functional Class is defined as: Class I: no limitation of physical activity, no symptoms of chest pain, angina, dyspnea, or near syncope with ordinary activity, Class II: slight limitation of physical activity, ordinary physical activity causes dyspnea or fatigue, chest pain, or near-syncope, Class III: marked limitation of physical activity, less than ordinary activity causes dyspnea or fatigue, chest pain, or near-syncope, and Class IV: inability to perform activity without symptoms, signs of right heart failure, dyspnea and or fatigue at rest, and discomfort is increased by any physical activity (Humbert et al., 2004). These screening tests will be counted as their baseline test results.

If the subjects meet criteria for enrollment they will return for the formal baseline visit approximately 14 days from screening. At this time they will have a 6MW/B followed by TT (unencouraged)>1 hr apart (Gomberg-Maitland et al., 2005; Patterson et al., 1972) a 2DE to assess TR velocity, a 3DE examination (experimental endpoint), and CT (experimental endpoint). Subjects will be seen weekly for safety evaluation with a clinical exam and documentation of WHO classification. Safety evaluation will include the following data: adverse events related to sorafenib toxicity, related to prostacyclin side effects, and related to phosphodiesterase inhibitor side effects. Each month, all subjects will have both a 6MW and TT as per protocol, laboratory including: CBC with differential, BCP (serum sodium), LFT (including albumin), uric acid, INR, BNP, troponin I, bFGF, and then be seen in the office to evaluate further dosing of sorafenib.

At month 4 subjects will be required to be seen on 2 consecutive days. Day 1 subjects will have an office visit, a baseline 6MW/Borg score, an assessment of WHO functional class, a TT, PFT, 2DE, 3DE, and CT. Day 2 will be a right heart catheterization. Hemodynamic values will be determined by serial measurements of hemodynamic parameters (specifically CO and mPAP) to demonstrate stability. Stable hemodynamics are defined by changes in CO and mPAP of less than or equal to 20% between three consecutive serial measurements at least 5 minutes apart. After hemodynamic stability is demonstrated, the hemodynamics and oxygen saturation variables from the last assessment will be recorded.

Safety endpoints at month 4 will include: time to clinical worsening defined as death, lung transplantation, hospitalization due to PH/right heart failure, a decrease in exercise measures by at least 20% compared with baseline measure, worsening hemodynamics: either an increase in mean PA pressure by >20%, an increase in PVR >20%, and or a decrease in CO by >20%, and worsening PFTs as evidence by a decrease in DLCO, FEV1, FVC, or TLC by >15%. Preliminary efficacy endpoints will include: monthly 6MW/B, WHO functional class, TT (experimental), 4 month right heart catheterization, TR velocity on 2DE, 3DE to assessRV parameters, (experimental), and a 64 slice CT (experimental).

Follow-Up: At the conclusion of 4 months, subjects with perceived benefit as evidenced by objective and subjective measures based on the determination of the principal investigator and the sponsors and advisors on the grant will continue on therapy at their month 4 dose up to 1 year of therapy. They will continue to have follow-up visits every 3 months with a 6MW test to a maximum of 1 year. They will be expected to speak with a member of the PH team to discuss AEs monthly by phone and all serious adverse events (SAE) will be reported to the PI and the IRB throughout the duration of the study.

B. Single Center Phase II Trial of Sorafenib in PAH Patients.

Objectives Phase II study: Objectives are to assess safety and efficacy endpoints of sorafenib by a randomized discontinuation design. This is a 2 center study with a placebo controlled study using a randomized discontinuation design: placebo or MTD based on Phase IB study

Accrual: Estimated 22-88 patients. Enrollment will be completed in 18-24 months—this will be based on preliminary efficacy from Phase IB.

Patient Selection: Eligibility and Exclusion Criteria as in Phase IB.

Treatment Plan:

Phase II will begin with a dose titration to the MTD based on the escalation described above; a 12-week open label run-in period. This will begin with a dose titration to the Phase IB MTD based on a titration regime of tolerance from the Phase IB study. If a subject does not tolerate any dose prior to the MTD or 1 month of MTD they will be withdrawn; not randomized into the placebo controlled study. Subjects with worsening 6MW>20% with worsening functional class or signs/symptoms of deterioration will be withdrawn from the study. After the 12-week run-in period, disease status will be assessed based on change in 6MW distance. Subjects with >50% improvement or >100 meter improvement will continue to receive sorafenib until disease progression or toxicity, in order to avoid potential ethical concerns about randomization of patients with apparent major clinical benefit. The inventors do not expect a withdrawal for clinical deterioration with sorafenib but since this occurs with prostacyclin it will be a potential risk. These patients will be followed as per standard of care every 3-6 months.

The remaining patients without obvious treatment benefit or failure during the open-label run-in period will be randomized in double-blind fashion using a central allocation via a telephone randomization system to receive the same dose of sorafenib or placebo. Evaluation of these subjects will be as per the Phase IB plan with weekly safety and monthly efficacy evaluations. Subjects with worsening 6MW>20% with worsening functional class or signs/symptoms of deterioration will be unblinded. If on placebo they will receive sorafenib at their previous dose; if on active drug they will be withdrawn. Those subjects who have a decline in 6MW without worsening clinical signs or symptoms will be seen at the next week visit and the 6MW will be repeated (unless there is evidence of clinical deterioration); this is to address the variability of the test and the variability of the individual. If the repeat 6MW still demonstrates deterioration, the subject will be unblinded to therapy and treatment will be as above: if on placebo they will receive sorafenib at their previous dose; if on active drug they will be withdrawn. DLT criteria will be followed as per Phase IB at weekly visits.

Data Collection:

Subjects will be followed as per Phase IB with the following differences: 1) If subjects have clinical deterioration at any time after randomization, defined as 6MW>20% decline with clinically assessed deterioration, or clinically assessed progression, they will be unblinded as per protocol. Subjects on placebo will be offered sorafenib at their previous dose, and subjects on active therapy, sorafenib will be withdrawn from study. 2) All subjects unblinded and started on active therapy will have a right heart catheterization after 6 months.

Withdrawal:

Subjects withdrawn from protocol due to clinical deterioration or DLT will have an office visit 30-40 days after end of treatment to record the following: physical exam, blood pressure, PH symptoms, WHO functional class, concomitant medications/AEs, dose of prostacyclin, 6MW and TT.

Statistical Analysis:

Based on the efficacy data: improvement in 6MW at 4 months on MTD a power calculation will be made to determine the sample size needed for this trial. The case report with combination therapy with imatinib at 3 and 6 months demonstrated a 60% improvement in 6MW. It is estimated that the sample size conservatively based on previous therapeutic trials to a maximum of 60% based on this report. A better estimate will be obtained based on Phase IB trial. For example, assuming a 30% improvement in 6MW, with 80% power, the estimated sample size is 39 subjects in each arm, a 40% improvement=23 subjects in each arm, and a 50% improvement=15 subjects in each arm and a 60% improvement=10 subjects in arm. The investigators will allow for a 10% drop-out rate and adjust the sample size accordingly. An early stopping rule, using the O'Brien Fleming alpha spending rule, using an α of 0.0003 leaving p=0.0497 after 35% of subjects enroll (subject to change based on efficacy from IB). The trial will be stopped if the rate of deterioration at 3 months after randomization is higher or lower than expected. The DSMB will determine if the trial need to be stopped.

Data Safety Monitoring Board:

The DSMB will consist of the Sponsor, Co-Sponsor, members of the Scientific Advisory Committee in addition to 2 outside PH specialists to be determined by the Principal Investigator (PI).

### Example 5

### Identification and Initiation of Early Clinical Trials of New Agents in PAH

The Clinical Therapeutics in Pharmacology and Pharmacogenomics group are informed of novel kinase inhibitors being evaluated by an oncology Phase I/II unit. As these agents are evaluated in oncology patients, they will be assessed for potential benefits and risks in PAH patients. If the agent scientifically has potential efficacy, the agent will be evaluated in preclinical studies with the hypoxic/SU5416 rat model of PAH.

Studies will be designed as done with sorafenib, with 15-18 male Dahl-sensitive rats weighing between 150 and 250 grams will be divided into 5 groups: normoxia+vehicle (normal, healthy control), hypoxia+vehicle (hypoxia control), hypoxia+SU-5416 (positive control), hypoxia+sorafenib (safety comparison with SU-5416), and hypoxia+sorafenib+SU-5416 (therapeutic activity assessment). All animal care and procedures will be preformed in accordance with institutional guidelines. Animals are housed in a Plexiglas chamber open to room air (normoxia) or maintained at 10% FiO2. Rats in the two SU-5416 groups will receive one injection of SU-5416 at the start of the experiment (20 mg/kg). Agent will be prepared for intraperitoneal lavage and dosed as per known rat protocols. Pressures and echocardiography will be preformed as described herein and in the scientific literature. If rats given the agent plus hypoxia do not develop PAH and appear to have some therapeutic activity (rats given SU5416 plus hypoxia, plus agent), the inventors will proceed to Phase IB study as described.

Phase IB studies will be designed as a small safety and tolerability study of the agent. The study will determine the MTD and assess vital signs and clinical exam assessment at weekly office visits. If the drug is tolerated with some preliminary efficacy, a Phase II study will be initiated. The study will be a randomized discontinuation trial. If Phase I and Phase II are not successful because subjects do not tolerate the medication based on severe DLT, or with minimal efficacy the process will be reinitiated with a novel therapeutic agent.

### Example 6

### Additional Phenotypic Markers

Treadmill test as new standard of care exercise measure. The inventors have begun creating a new model using the data from the intravenous treprostinil study, based on the preliminary data demonstrating that the treadmill test (TT) was similar to the 6MW, had repeated reliability, and predicted functional class. The relationship between distance (DIST) and MET was modeled using generalized estimating equation (GEE) models which accounted for the within patients correlations since multiple measurements were conducted for each patient. The inventors first fitted a piecewise regression using data between baseline and month 3 (Model 1) and validated the model fitting using data between month 6 and month 12. The data had a “good” correlation, a “good fit” and thus the inventors were able to develop a piecewise regression model with all data (Model 2). The new model appears to differentiate less sick patients and is as good as the 6MW.

To complete analyses, the inventors will develop receiver operator curves accounting for repeated measures to compare the 6MW to the TT. Based on this analysis, if TT is as good as 6MW, logistic regression analyses will be done on this data set to determine if MET is a good predictor of clinical worsening, survival, and clinical outcome variables including hemodynamics, echocardiography, and laboratory biomarkers. The model will estimate predicted MET based on 6MW distance but does not predict MET based on the patient's individual characteristics. At the University of Chicago, all patients have yearly TT evaluations, and evaluations pre and post medication changes. Initially, the model will be assessed in the University of Chicago dataset of patients having both the 6MW and the TT. A prediction equation will be developed for the TT to determine MET expected for individual PH patient based on WHO class. In addition, the inventors will be using the TT as an experimental endpoint in all trials conducted.

TABLE 4

| | | Model 1: | | Model 2: | |
|---|---|---|---|---|---|
| | | Piecewise regression | | Piecewise regression | |
| | | with data 0-3 months | | with data 0-12 months | |
| | | (n = 127) | | (n = 171) | |
| | | slope < 4 107.1795 | | slope < 4 110.3069 | |
| Observed data (all | | slope ≧ 4 19.12348 | | slope ≧ 4 19.37262 | |
| data 0-12 months | | intercept −31.6585 | | Intercept −42.90939 | |
| MET | mean DIST | MET | DIST | MET | DIST |
| 2 | 176.0 | 2 | 183 | 2 | 178 |
| 3 | 288.4 | 3 | 290 | 3 | 288 |
| 4 | 395.8 | 4 | 397 | 4 | 398 |
| 5 | 415.1 | 5 | 416 | 5 | 418 |
| 6 | 442.2 | 6 | 435 | 6 | 437 |
| 7 | 460.1 | 7 | 454 | 7 | 456 |
| 8 | 476.9 | 8 | 474 | 8 | 476 |
| 9 | 423.5 | 9 | 493 | 9 | 495 |
| 10 | 513.8 | 10 | 512 | 10 | 515 |
| 11 | 545.5 | 11 | 531 | 11 | 534 |

3DE and CT scans as novel non-invasive measures. Continued preliminary data will be obtained for both technologies. As development progresses, these measures will be linked with clinical phenotype obtained from the PHC database. Surprisingly on 3DE the tricuspid valve annulus does not dilate as expected with increasingRV size. Measures of RV 3D areas will be compared with 2D imaging using Spearman rank correlations. Change in area as a response to therapy will be recorded. TR velocity will be measured in 2DE. All of these measures will be evaluated based on clinical characteristics, response to therapy, and outcomes to determine predictive value. Use of this modality in the evaluation of VEGF inhibitors and other vascular signaling targets on the right ventricle and pulmonaiy artery in our early drug development studies should prove to be a useful endpoint. For CT scans, the University of Chicago's 64 detector CT scan will be used to implement routine chest CT on all PH patients. The inventors contemplate using automated technology and advanced detection to identify plexiform lesions and novel indices of PH severity. The goal is to detect and then use the technology to follow response to therapy and as a possible screening tool.

Biomarker development. Blood samples will be obtained from all patients seen by the PH program and stored for future evaluation of potential serum markers, genetic, and proteomic evaluation. Concurrent with Phase I/II trials, biomarkers previously described will be assessed: LFT (albumin), uric acid, serum sodium, BNP, troponin I, bFGF, angiopoietin-2. Response to therapy may also be linked to genetic profiles. The GCRC will help with the processing and storage of samples. A database has been created compliant with HIPPA standards.

### Example 7

### Sorafenib Relieves Pulmonary Pressure

A 3 week study was designed and executed to assess the safety and therapeutic activity of sorafenib in a hypoxia-induced model of pulmonary hypertension in Dahl SS rats. A compound with a reportedly more limited spectrum of kinase inhibitory activity—SU5416 was previously demonstrated to exacerbate pulmonary hypertension in the Dahl rat hypoxemic model. As part of the spectrum of sorafenib's pharmacologic activity overlaps with SU5416, we performed this study to determine whether sorafenib had similar effects or with additional mechanisms of inhibitory activity could counteract the effects of SU5416.

Fifteen Dahl Salt Sensitive strain rats were divided into 5 groups: normoxia+vehicle (normal, healthy control), hypoxia+vehicle (hypoxia control), hypoxia+SU5416 (positive control), hypoxia+sorafenib (safety comparison with SU5416), and hypoxia+sorafenib+SU5416 (therapeutic activity assessment). Echocardiograms were performed on all rats at the start of the experiment. Except for the normoxia group, all rats were maintained in a hypoxia chamber with a partial pressure of oxygen of 10% FiO2 for the duration of the experiment. Rats in the 2 SU5416 groups received one subcutaneous injection of SU5416 at the start of the experiment (20 mg/kg). Stock sorafenib solutions were prepared every three days, crushing and dissolving sorafenib tablets in EL/ethanol (50:50; Sigma Cremophor EL, 95% ethyl alcohol) at final concentration of 4 mg/mL, protected from light exposure and stored at room temperature. Final dosing solutions were prepared on the day of use by dilution of the stock solution to 1 mg/mL with water and administered by gavage to the rats daily. After 3 weeks, all rats had echocardiography and hemodynamic studies. Organ and blood specimens were obtained for further evaluation.

As has been previously described, rats given SU5416 developed pulmonary hypertension measured by elevated right ventricular and pulmonary artery pressures, echocardiographic changes, and elevated right ventricle/left ventricular weights. Rats exposed to hypoxia had mildly elevated pressures compared with normoxia and there was no significant change in pressures or weights in rats given hypoxia plus sorafenib. Sorafenib appears to have a beneficial effect on pulmonary hypertensive rats as rats in hypoxia, plus SU5416, plus sorafenib had pressures and weights similar to normoxia. Table 4. The small sample size did not allow for significant change in echocardiographic data but there appeared to be a positive trend with this combination.

Sorafenib appears to protect SU5416 treated hypoxia-exposed rats from pulmonary arterial hypertension. Furthermore, while sorafenib and SU5416 share some pharmacological activity, unlike SU5416, sorafenib does not exacerbate hypoxia-induced hypertension. Based on these results the inventors repeated the experiment with 18 rats distributed in the same 5 groups (only difference was 3 additional rats in the normoxia control group). The results were reproducible. In the initial experiment two rats died during induction of anesthesia (after unblinding normoxia, Hypoxia/SU5416). In the subsequent experiment, one rat died on day zero (after unblinding hypoxia/SU5416).

Sorafenib appears to protect SU5416-treated hypoxia-exposed rats from pulmonary arterial hypertension. Furthermore, while sorafenib and SU5416 share some pharmacological activity, unlike SU5416 sorafenib does not exacerbate hypoxia-induced hypertension.

TABLE 5
Preliminary echocardiogram findings

| | Wt (g) | RVAW | LVPW | FS (%) | LV Mass (gram) | CO (Ao) |
|---|---|---|---|---|---|---|
| Normoxia-PRE | 337 ± 19 | 0.14 | 0.24 ± 0.06 | 57 ± 10 | 1.57 ± 0.06 | 500 ± 29 |
| (n = 3) | | | | | | |
| Normoxia- | 386 ± 6 | 0.14 | 0.24 ± 0.02 | 68 ± 4 | 1.40 ± 0.00 | 387 ± 30 |
| POST (n = 2) | | | | | | |
| hypoxia-PRE | 350 ± 14 | | 0.24 ± 0.10 | 79 ± 3 | 1.50 ± 0.44 | 397 ± 36 |
| (n = 3) | | | | | | |
| hypoxia-POST | 353 ± 25 | 0.14 | 0.16 ± 0.02 | 74 ± 11 | 1.08 ± 0.13 | 323 ± 128 |
| (n = 3) | | | | | | |
| SU5416 + hypoxia- | 328 ± 11 | 0.07 | 0.16 ± 0.04 | 71 ± 11 | 1.30 ± 0.14 | 310 ± 10 |
| PRE (n = 2) | | | | | | |
| SU5416 + hypoxia- | 324 ± 22 | 0.16 ± 0.03 | 0.16 ± 0.02 | 63 ± 4 | 1.25 ± 0.33 | 261 ± 55 |
| POST (n = 3) | | | | | | |
| sorafenib-PRE | 328 ± 10 | 0.11 | 0.21 ± 0.03 | 78 ± 5 | 1.60 ± 0.30 | 577 ± 418 |
| (n = 3) | | | | | | |
| sorafenib-POST | 331 ± 15 | | 0.18 ± 0.03 | 68 ± 20 | 1.30 ± 0.10 | 326 ± 246 |
| (n = 3) | | | | | | |
| sorafenib + SU5416- | 332 ± 3 | 0.09 | 0.22 ± 0.04 | 71 ± 11 | 1.43 ± 0.21 | 247 ± 65 |
| PRE (n = 3) | | | | | | |
| sorafenib + SU5416- | 318 ± 13 | 0.11 ± 0.01 | 0.20 ± 0.04 | 75 ± 14 | 0.93 ± 0.05 | 216 |
| POST (n = 3) | | | | | | |

| | | | | | | TV max |
|---|---|---|---|---|---|---|
| | | | PA PG | | | PG |
| | CI (Ao) | MPA | (mmHg) | CO (PA) | CI(PA) | (mmHg) |
| Normoxia-PRE | 1.49 ± 0.11 | 0.45 ± 0.05 | 7.47 ± 3.97 | 736 ± 478 | 2.23 ± 1.55 | |
| (n = 3) | | | | | | |
| Normoxia- | 1.00 ± 0.09 | 0.43 ± 0.05 | 8.75 ± 0.64 | 544 ± 130 | 1.41 ± 0.32 | 8.80 ± 5.94 |
| POST (n = 2) | | | | | | |
| hypoxia-PRE | 1.14 ± 0.15 | 0.40 ± 0.03 | 5.07 ± 2.20 | 406 ± 115 | 1.16 ± 0.30 | 6.80 |
| (n = 3) | | | | | | |
| hypoxia-POST | 0.91 ± 0.35 | 0.40 ± 0.03 | 4.43 ± 1.78 | 398 ± 85 | 1.12 ± 0.16 | 2.30 ± 0.71 |
| (n = 3) | | | | | | |
| SU5416 + hypoxia- | 0.95 ± 0.06 | 0.45 ± 0.01 | 3.65 ± 2.76 | 400 ± 99 | 1.21 ± 0.26 | |
| PRE (n = 2) | | | | | | |
| SU5416 + hypoxia- | 0.82 ± 0.22 | 0.41 ± 0.05 | 2.05 ± 0.92 | 279 ± 69 | 0.87 ± 0.14 | 5.63 ± 4.07 |
| POST (n = 3) | | | | | | |
| sorafenib-PRE | 1.77 ± 1.30 | 0.48 ± 0.07 | 6.10 ± 5.52 | 551 ± 467 | 1.70 ± 1.43 | |
| (n = 3) | | | | | | |
| sorafenib-POST | 1.00 ± 0.80 | 0.38 ± 0.07 | 3.87 ± 0.95 | 271 ± 48 | 0.82 ± 0.17 | 4.85 ± 3.46 |
| (n = 3) | | | | | | |
| sorafenib + SU5416- | 0.74 ± 0.19 | 0.43 ± 0.01 | 2.00 ± 1.18 | 313 ± 166 | 0.94 ± 0.49 | |
| PRE (n = 3) | | | | | | |
| sorafenib + SU5416- | 0.65 | 0.45 ± 0.04 | 3.80 ± 1.41 | 359 ± 106 | 1.12 ± 0.27 | |
| POST (n = 3) | | | | | | |

### REFERENCES

The following references, to the extent that they provide exemplary procedural or other details supplementary to those set forth herein, are specifically incorporated herein by reference.
U.S. Pat. No. 5,145,684
U.S. Pat. No. 5,399,363
U.S. Pat. No. 5,466,468
U.S. Pat. No. 5,543,158
U.S. Pat. No. 5,552,157
U.S. Pat. No. 5,565,213
U.S. Pat. No. 5,567,434
U.S. Pat. No. 5,580,579
U.S. Pat. No. 5,641,515
U.S. Pat. No. 5,641,515
U.S. Pat. No. 5,725,871
U.S. Pat. No. 5,738,868
U.S. Pat. No. 5,741,516
U.S. Pat. No. 5,756,353
U.S. Pat. No. 5,780,045
U.S. Pat. No. 5,792,451
U.S. Pat. No. 5,795,587
U.S. Pat. No. 5,804,212
U.S. Patent Pub. 2001/0027202
U.S. Patent Pub. 2003/0125359
U.S. Patent Pub. 2003/0139605
Allen and Chonn, FEBS Lett., 223(1):42-6, 1987.
Arcasoy et al., Am J Respir Crit. Care Med 167:735-40, 2003
ATS statement: guidelines for the six-minute walk test. Am J Respir Crit. Care Med 166:111-7, 2002
Badesch et al., Am. Coll. Cardiol., 43(12 Suppl S):56S-61S, 2004.
Balasubramaniam et al., Am J Physiol Lung Cell Mol Physiol 284:L826-33, 2003
Barst et al., N Engl J Med 334:296-301, 1996
Barst, J Clin Invest 115:2691-4, 2005
Benet et al, Goodman and Gilman's The Pharmacological Basis Of Therapeutics, Hardman et al (Eds.), 9^{th} Ed., Chap. 1, McGraw-Hill, NY, 1996.
Benisty et al., Chest 126:1255-61, 2004
Biomarkers and surrogate endpoints: Preferred definitions and conceptual framework.

Clinical Pharmacology and Therapeutics (CPT) 69:89-95, 2001
Chalidran et al., Indian J Exp Biol., 35(8):801-809, 1997.
Chu et al., Ann Thorac Surg 77:449-56, 2004
Clapp et al., Am J Respir Cell Mol Biol 26:194-201, 2002
Cool et al., Am JPathol 155:411-419, 1999
Couvreur et al., Bull. Mem. Acad. R. Med. Belg., 143(7-9):378-388, 1988.
Couvreur et al., FEBS Lett., 84(2):323-326, 1977.
Couvreur et al., J. Pharm. Sci., 69(2):199-202, 1980.
Couvreur, Crit. Rev. Ther. Drug Carrier Syst., 5(1):1-20, 1988.
Cross et al., Trends Biochem Sci, 28:488-494, 2003.
Czeslick et al., Eur J Clin Invest 33:1013-7, 2003
D'Alonzo et al., Ann Intern Med 115:343-9, 1991
Data et al., Circulation 64:4-12, 1981
Dorfmuller et al., Eur Respir J 22:358-63, 2003
Douglas et al., Crit. Rev Ther Drug Carrier Syst, 3(3):233-61, 1987.
Eddahibi et al., J Clin Invest 2001 108:1141-50, 2001
Farber and Loscalso, N. Engl. J. Med., 351:1655-1665, 2004.
Friede A, Grossman R, Hunt R, Li R, Stern S: National Biospecimen Network Blueprint, in Constella Group I (ed). Durham, N.C., 2003
Frost et al., Vascul Pharmacol 43:36-9, 2005
Gabizon and Papahadjopoulos, Proc. Natl. Acad. Sci. USA, 85(18):6949-6953, 1988.
Galie et al., N Engl J Med 353:2148-57, 2005
Geraci et al., J Clin Invest 103:1509-15, 1999
Ghofrani et al., J Am Coll Cardiol 42:158-64, 2003
Ghofrani et al., N Engl J Med 353:1412-3, 2005
Giaid and Saleh, New Engl J Med 333:214-21, 1995
Giaid et al., New Engl J Med 328:1732-9, 1993
Giaid, Chest 114:208 S-212S, 1998
Giannelli et al., Curr Med Res Opin 21:327-32, 2005
Girgis et al., Am J Physiol Heart Circ Physiol 285:H938-45, 2003
Girgis et al., Am J Respir Crit. Care Med 172:352-7, 2005
Gomberg-Maitland et al., Am J Respir Crit. Care Med 172:1586-9, 2005
Gomberg-Maitland et al., Am J Cardiol 96:1334-6, 2005
Henry-Michelland et al., Int. J. Pharm., 35:121-127, 1987.
Herve et al., Am J Med 99:249-254, 1995
Hirahata et al., Gan To Kagaku Ryoho, 19(10):1591, 1992.
Hoeper et al., N Engl J Med 342:1866-70, 2000
Homma et al., J Heart Lung Transplant 20:833-9, 2001
Humbert et al., N Engl J Med 351:1425-36, 2004
Hwang et al., Crit. Rev. Ther. Drug Carrier Syst., 15(3):243-284, 1998.
Itoh et al., Am J Respir Crit. Care Med 169:34-8, 2004
Kawut et al. Am J Cardiol 95:199-203, 2005
Kawut et al., Chest 128:2355-62, 2005
Kido et al., J Thorac Cardiovasc Surg 129:268-76, 2005
Kopec et al., J Clin Epidemiol 46:959-71, 1993
Kucher et al., Arch Intern Med 165:1777-81, 2005
Laliberte et al., J Cardiovasc Pharmacol 44:209-14, 2004
Lasic, Trends Biotechnol., 16(7):307-321, 1998.
Lee et al., J Clin Invest 101:927-934, 1998
Leuchte et al., Chest 128:2368-74, 2005
Leuchte et al., J Am Coll Cardiol 43:764-70, 2004
Marcos et al., Circ Res 94:1263-70, 2004
Margalit, Crit. Rev. Ther. Drug Carrier Syst., 12(2-3):233-261, 1995.
Mathiowitz et al., Nature, 386(6623):410-414, 1997.
McLaughlin et al., Circulation 106:1477-82, 2002
McLaughlin et al., Chest, 126(1):78S-92S, 2004.
Morishita et al., Proc. Natl. Acad. Sci. USA, 90:8474, 1993.
Nagaya et al., Am J Respir Crit. Care Med 160:487-492, 1999
Nagaya et al., Circulation 102:2005-10, 2000
Nauser and Stites, Am. Fam. Physician., 63(9):1789-1798, 2001.
Olschewski et al., N Engl J Med 347:322-9, 2002
Oudiz and Ginzton, J Heart Lung Transplant 22:832-3, 2003
Oudiz et al., Am J Cardiol 97:123-126, 2006
Park et al., Congest Heart Fail 10:221-5, 2004
Patterson et al., Am J Cardiol 30:757-62, 1972
Petrova et al. Exp Cell Res, 253:117-130, 1999
Physician's Desk Reference 2003.
Pinto-Alphandary et al., J. Drug Target, 3(2):167-169, 1995.
Quintanar-Guerrero et al., Pharm. Res., 15(7):1056-1062, 1998.
Remington's Pharmaceutical Sciences” 18^{th} Ed., 1545-1568, 1990.
Remington's Pharmaceutical Sciences” 18^{th} Ed., 1596-1609, 1990.
Remington's Pharmaceutical Sciences, 18th Ed. Mack Printing Company, 711, 1990.
Rich, Advances in Pulmonary Hypertension, 1(1):3, 2002.
Rich, Ann. Intern. Med., 107(2):216-223, 1987.
Rondelet et al., Circulation 110:2220-5, 2004
Rosner et al., J Clin Oncol 20:4478-84, 2002
Rubin et al., Expert Opinion 11:991-1002, 2002
Rubin, Advances in Pulmonary Hypertension, 1(1):16 and 19, 2002
Rubin, Chest, 126(1):4S-6S; 7S-10S, 2004.
Rubin, The New England Journal of Medicine, 336(2):111, 1997.
Schermuly et al., J Clin Invest 115:2811-21, 2005
Simonneau et al., Am J Respir Crit. Care Med 165:800-4, 2002
Sitbon et al., J Am Coll Cardiol 40:780-8, 2002
Somiari et al., Transactions of the Integrated Biomedical Informatics & Enabling
Technologies Symposium Journal 1:131-143, 2004
Stadler et al., J Clin Oncol 23:3726-32, 2005
Stella et al., In: Prodrugs: A Chemical Approach to Targeted Drug Delivery, Borchardt et al. (Ed.), Humana Press, 247-267, 1985.
Stiebellehner et al., Chest 123:1293-5, 2003
Strumberg et al., J Clin Oncol 23:965-72, 2005
Sullivan et al., Proc Natl Acad Sci USA 100:12331-12336, 2003
Takakura, Nippon Rinsho, 56(3):691-695, 1998.
Takenaga et al., J Control Release, 52(1-2):81-87, 1998.
Tapson, Advances in Pulmonary Hypertension, 1(1): 16-17, 2002
Taraseviciene-Stewart et al., FASEB J 15:427-38, 2001
The Merck Manual, 17^{th} Ed., 595, 1999.
Torbicki et al., Circulation 108:844-8, 2003
Tuder et al., Am S Pathol 144:275-285, 1994
Tuder et al., Am S Respir Crit. Care Med 159:1925-1932, 1999
Tuder et al., J Pathol 195:367-74, 2001
Tuder, Semin Respir Crit. Care Med 15:207-214, 1994
Ushio-Fukai et al., Arterioscler Thromb Vasc Biol 21:489-95, 2001
Wade et al., J Clin Pharmacol 44:83-8, 2004
Wagenvoort and Mulder, Chest:844-849, 1993
Wagenvoort and Wagenvoort, Pathology of pulmonary hypertension (ed 2nd). New York, John Wiley and Sons, 1977
Welsh et al., Chest 110:710-717, 1996
Wilhelm et al., Cancer Res 64:7099-109, 2004
Wilman, In: Prodrugs in Cancer Chemotherapy, Biochemical Society Transactions, 14:375-382, Belfast, 1986.

Xin et al., Biochem Biophys Res Commun 204:557-64, 1994
Yasunobu et al., Chest 127:1637-46, 2005
Zambaux et al. J Protein Chem., 17(3):279-284, 1998.
zur Muhlen et al., Eur. J. Pharm. Biopharm., 45(2):149-155, 1998.

1. A method comprising providing sorafenib to a subject with pulmonary arterial hypertension (PAH), with symptoms of PAH, or at risk for PAH.

2. The method of claim 1, wherein the subject is provided with sorafenib by administering or prescribing to the subject sorafenib, a sorafenib prodrug, or a pharmaceutically acceptable salt thereof.

3. The method of claim 1, further comprising identifying a subject with PAH or symptoms of PAH.

4. The method of claim 3, wherein the subject is diagnosed as having severe PAH.

5. The method of claim 1, wherein the patient has a mean pulmonary artery pressure equal to or greater than 25 mm Hg with a pulmonary capillary or left atrial pressure equal to or less than 15 mm Hg.

6. The method of claim 1, further comprising evaluating PAH in the subject.

7. The method of claim 6, wherein the patient is evaluated before and/or after administering the composition.

8. The method of claim 6, wherein the subject is evaluated for PAH by having an electrocardiogram, an echocardiogram, pulmonary function tests (PFTs), a perfusion lung scan, and/or a right-heart cardiac catheterization.

9. The method of claim 1, wherein the subject is provided multiple doses of sorafenib.

10. The method of claim 9, wherein a dose is between about 5 and about 500 mg of sorafenib or a sorafenib prodrug.

11. The method of claim 2, wherein the sorafenib or sorafenib prodrug is administered or prescribed for administration orally, intravenously, intraarterially, inhalation.

12. The method of claim 1, further comprising providing at least a second PAH treatment.

13. The method of claim 12, wherein the second treatment is an anticoagulant, an calcium channel blocker, a prostacyclin, Bosentan, nitric oxide, Sildenafil, a diuretic, a cardiac glycoside, a vasodilator, an endothelin antagonist, a phosphodiesterase inhibitor, an endopeptidase inhibitor, a lipid lowering agent, a thromboxane inhibitor, or oxygen.

14. The method of claim 1, wherein the sorafenib is provided before, after, or during surgery.

15. The method of claim 14, wherein the surgery comprises lung transplantation.

16. A method of reducing pressure in the pulmonary artery of a patient comprising administering to the patient an effective amount of a composition comprising sorafenib, a sorafenib prodrug, or a pharmaceutically acceptable salt thereof.

17. A method for treating or preventing PAH in a patient comprising administering to the patient an effective amount of sorafenib, a sorafenib prodrug, or a pharmaceutically acceptable salt thereof.

18. A pharmaceutical composition comprising (a) sorafenib or a sorafenib prodrug and (b) at least a second PAH treatment.

19. The pharmaceutical composition of claim 18, wherein the second PAH treatment comprises an anticoagulant, an calcium channel blocker, a prostacyclin, Bosentan, nitric oxide, Sildanefil, a diuretic, a cardiac glycoside, a vasodilator, an endothelin antagonist, a phosphodiesterase inhibitor, an endopeptidase inhibitor, a lipid lowering agent, or a thromboxane inhibitor.

20. A method comprising providing a VEGFR2 inhibitor to a subject with pulmonary arterial hypertension (PAH), with symptoms of PAH, or at risk for PAH.

21. The method of claim 20, wherein the VEGFR2 inhibitor is selected from abt-869, amg706, AZD2171, bay57-9352, sorafenib, XL647, XL999, GW786034, bevacizumab, PKC412, AEE788, PTK787 (vatalanib), OSI-930, OSI-817, SU11248, AG-013736, ZK3-4709, quinazoline ZD6474, pyrrolocarbazole CEP-7055, or CP-547632.
